SEQUENCE LISTING (5 sequences)

SEQ ID NO: 1 - DNA, 21 bp, Rattus norvegicus
  misc_feature (1)..(21): Forward primer SRY gene
agaggcacaagttggctcaac

SEQ ID NO: 2 - DNA, 22 bp, Rattus norvegicus
  misc_feature (1)..(22): Reverse primer SRY gene
tcccactgatatcccagctgct

SEQ ID NO: 3 - DNA, 28 bp, Rattus norvegicus
  misc_feature (1)..(28): FAM-TAMRA SRY gene Probe
caacagaatcccagcatgcagaattcag

SEQ ID NO: 4 - DNA, 20 bp, Rattus norvegicus
  misc_feature (1)..(20): Forward Primer 2 SRY Gene
ggagagaggcacaagttggc

SEQ ID NO: 5 - DNA, 19 bp, Rattus norvegicus
  misc_feature (1)..(19): Reverse Primer 2 SRY Gene
tcccagctgcttgctgatc

(19) United States
(12) Patent Application Publication
(10) Pub. No.: US 2015/0010640 A1
(43) Pub. Date: Jan. 8, 2015

(54) BI-FUNCTIONAL COMPOSITIONS FOR TARGETING CELLS TO DISEASED TISSUES AND METHODS OF USING SAME

(71) Applicant: Cedars-Sinai Medical Center, Los Angeles, CA (US)

(72) Inventors: Eduardo Marban, Beverly Hills, CA (US); Ke Cheng, Raleigh, NC (US)

(21) Appl. No.: 14/333,440

(22) Filed: Jul. 16, 2014

Related U.S. Application Data
(63) Continuation of application No. PCT/US2013/039255, filed on May 2, 2013.
Application No. PCT/US2013/039255 is a continuation-in-part of application No. 13/504,747, filed on Apr. 27, 2012.
(60) Provisional application No. 61/641,784, filed on May 2, 2012.
(60) Provisional application No. 61/255,438, filed on Oct. 27, 2009.

(51) Int. Cl. A61K 47/48; A61K 45/06

(52) U.S. Cl. CPC A61K 47/48861; A61K 45/06; A61K 47/48561
USPC 424/491; 424/178.1; 424/179.1; 424/93.7

(57) ABSTRACT

Disclosed herein are compositions and methods for the targeted delivery of therapeutic cells to a target tissue. In several embodiments, the therapeutic cells are captured by an antibody that is coupled to a magnetic particle, which is in turn coupled to an antibody directed against a specific marker expressed by a target tissue. In some embodiments, the therapeutic cells comprise the target tissue is damaged or diseased cardiac tissue. In several embodiments, in conjunction with an applied magnetic field, the methods, in combination with the compositions, yield enhanced delivery, of the therapeutic cells to the target tissue, thereby resulting in repair and/or regeneration of the target tissue. Also disclosed are methods for the non-invasive detection of immune responses to transplanted cells or organs.

### CROSS-REFERENCE TO RELATED APPLICATIONS

This application is a continuation of International Application No. PCT/US2013/039255, filed May 2, 2013, which claims the benefit of U.S. Provisional Application No. 61/641,784, filed on May 2, 2012. This application is also a continuation-in-part of U.S. application Ser. No. 13/504,747, filed Apr. 27, 2012, which is the United States National Phase Application under 35 U.S.C. §371 of International Application No. PCT/US2010/054358, filed Oct. 27, 2010, which claims the benefit of U.S. Provisional Application No. 61/255,438, filed on Oct. 27, 2009. The entire disclosure of each of the applications listed above is incorporated by reference herein.

### STATEMENT REGARDING FEDERALLY SPONSORED RESEARCH

This invention was made with Government support under Department of Defense Congressionally Directed Medical Research Program/Peer Reviewed Medical Research Program Investigator-Initiated Research Award #PR120246. The Government has certain rights in the invention.

### BACKGROUND

1. Field of the Invention

Several embodiments of the present application relate generally to compositions and methods for the capture of endogenous or exogenous cells and enhanced delivery and retention of those captured cells to a diseased target tissue. In several embodiments, the compositions provided herein, and methods of using same, provide improved methods for treating tissue degeneration, cancer and infectious diseases (or otherwise damaged tissues), without the need for the generation of exogenous populations of cells, as well as novel diagnostic techniques.

2. Description of the Related Art

Heart disease is a leading cause of fatalities in modern societies. In recent years, the use of stem cells has offered tremendous potential for treating various cardiac diseases. Numerous other diseases (including those specific to certain organs) are also the subject of various attempts at stem cell therapy. A variety of cell therapy approaches exist that aim to restore function to or replace damaged or diseased tissues.

### SUMMARY

Two major issues to be addressed by traditional cell therapies are (i) having a sufficient number of cells to be used in therapy at any given time and (ii) how to specifically target and/or retain the cells at a target tissue. For example, retention rate of exogenously-delivered therapeutic cells in the heart can be low due to the wash-out effect caused by blood flow, coupled with extrusion of injected cells at the injection site due to normal contraction of the heart. Accordingly, there is a need in the art to provide compositions and methods that provide targeted cell delivery with enhanced cell delivery, engraftment, and/or retention.

Given the need to improve targeted cell delivery and enhanced therapeutic effects, there is provided, in several embodiments, a method for treating damaged or diseased tissue comprising administering to a subject having damaged or diseased tissue a composition comprising magnetic particles coupled to a first population of antibodies and a second population of antibodies, and applying a magnetic field around or adjacent to the damaged or diseased tissue. In several embodiments, the magnetic field enhances the targeting of the composition to the damaged or diseased tissue, and/or counteracts the wash-out of the composition from the damaged or diseased tissue. In several embodiments, the first population of antibodies is directed to a marker expressed by population of therapeutic cells and the second population of antibodies is directed to a marker expressed by the damaged or diseased tissue of the subject. In several embodiments, the enhanced targeting associated with the magnetic field also enhances the interaction between the second population of antibodies and the markers expressed by the damaged or diseased tissue, thereby enhancing the delivery of the therapeutic cells to the damaged or diseased tissue. As a result, the enhanced delivery of the therapeutic cells provides therapeutic improvements in the damaged or diseased tissue, thereby treating the damaged or diseased tissue.

In several embodiments, the damaged or diseased tissue comprises damaged or diseased cardiac tissue and the second population of antibodies is directed to a marker expressed by damaged or diseased cardiac tissue. In several embodiments, the first population of antibodies is selected from the group consisting of antibodies directed against CD34, antibodies directed against c-kit and antibodies directed against CD45 (and combinations thereof) and in several embodiments the second population of antibodies is selected from the group consisting of antibodies directed against myosin light chain, antibodies directed against IL-1 beta, IL-6, IL-8, and VEGFR-2 (or combinations thereof). In several embodiments, the marker targeted by the second population of antibodies is myosin light chain. In several embodiments, the first population of antibodies is directed to the CD34 marker expressed on stem cells.

In several embodiments, the cardiac tissue has been damaged by an acute adverse cardiac event, such as an ischemic event, myocardial infarction (or multiple infarctions), trauma, coronary (or other) arterial occlusion, etc. In several embodiments, the damaged cardiac tissue results from chronic stress or disease of the heart, such as, for example, chronic heart failure, systemic hypertension, pulmonary hypertension, valve dysfunction, congestive heart failure, coronary artery disease, or combinations thereof. Combinations of acute and chronic events may also give rise to damaged or diseased cardiac tissue.

The therapeutic benefit of the methods and compositions disclosed herein are potentially multifold. In several embodiments, the therapeutic improvements comprise functional or anatomical repair of the damaged or diseased cardiac tissue. Functional improvement is realized, in several embodiments, by an increase in cardiac output and/or an increase in left ventricular ejection fraction. In several embodiments, the left ventricular ejection fraction is increased by at least 2%. In several embodiments, the therapeutic improvement comprises anatomical repair of the damaged or diseased tissue. In several embodiments, this repair comprises an increase in viable cardiac tissue. In several embodiments, anatomical repair comprises an increase in cardiac wall thickness. In several embodiments, anatomical repair comprises a decrease in scar tissue formation. In several embodiments, one or more types of anatomical repair are realized in conjunction with one or more functional improvements. However, in several embodiments, a functional improvement(s) is realized without an associated anatomical repair(s) and, in several embodiments, an anatomical repair(s) is realized without an associated functional improvement(s).

In several embodiments, the population of therapeutic cells is endogenous to the subject. However, in several embodiments the population of therapeutic cells is exogenous to the subject. Combinations of endogenous and exogenous cells are used, in several embodiments.

In several embodiments, the magnetic particles comprise superparamagnetic iron oxides (SPIO). In several embodiments, the magnetic particles have a diameter of about 10 to about 10,000 nanometers. In several embodiments, the magnetic particles are covalently coupled to the first and second populations of antibodies. In several embodiments, the covalent coupling is achieved by modification of carboxyl groups coating the magnetic particles. After coupling, depending on the embodiment, the magnetic particles have a diameter of about 30 to 15000 nanometers.

In several embodiments, the magnetic composition is delivered systemically, such as for example by a route selected from intravenous, intra-arterial, intracoronary, and/or intraventricular administration.

In several embodiments, the first population of antibodies recognizes CD45 on stem cells. In several embodiments, the stem cells are bone marrow stem cells.

In several embodiments, the first population of antibodies recognizes a population of immune cells selected from the group consisting of tumor-infiltrating lymphocytes, natural killer cells, cytotoxic T cells, T helper cells, T regulatory cells, and antigen presenting cells.

In several embodiments, the damaged or diseased tissue comprises a cancerous tissue. Depending on the embodiment, the cancerous tissue is affected with one or more cancers selected from the group consisting of acute Lymphoblastic Leukemia (ALL), Acute Myeloid Leukemia (AML), Adrenocortical Carcinoma, Kaposi Sarcoma, Lymphoma, gastrointestinal cancer, appendix Cancer, Central Nervous System cancer, basal Cell Carcinoma, Bile Duct Cancer, Bladder Cancer, Bone Cancer, Brain Tumors (including but not limited to Astrocytomas, Spinal Cord Tumors, Brain Stem Glioma, Craniopharyngioma, Ependymoblastoma, Ependymoma, Medulloblastoma, Medulloepithelioma, Breast Cancer, Bronchial Tumors, Burkitt Lymphoma, cervical cancer, colon cancer, Chronic Lymphocytic Leukemia (CLL), Chronic Myelogenous Leukemia (CIVIL), Chronic Myeloproliferative Disorders, ductal carcinoma, endometrial cancer, esophageal cancer, gastric cancer, hodgkin lymphoma, hairy cell leukemia, renal cell cancer, leukemia, oral cancer, liver cancer, lung cancer, lymphoma, melanoma, ocular cancer, ovarian cancer, pancreatic cancer, prostate cancer, pituitary cancer, uterine cancer, and vaginal cancer.

In several embodiments, the damaged or diseased tissue comprises infected tissue. The infection may be caused by an infectious agent selected from the group consisting of bacteria, fungi, viruses, and combinations thereof. In such embodiments, the therapeutic improvements comprise one or more of the inhibition, removal, or elimination of the infectious agent. In such embodiments, the population of therapeutic cells is a population of immune cells selected from the group consisting of neutrophils, monocytes, macrophages, dendritic cells, mast cells, epithelial cells, endothelial cells, fibroblasts, and mesenchymal cells. In several embodiments, the tissue is infected with one or more bacteria selected from the group of genera consisting of Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, and Yersinia. In several embodiments, the tissue is infected with one or more viruses selected from the group consisting of adenovirus, Coxsackievirus, Epstein-Barr virus, hepatitis a virus, hepatitis b virus, hepatitis c virus, herpes simplex virus, type 1, herpes simplex virus, type 2, cytomegalovirus, ebola virus, human herpesvirus, type 8, HIV, influenza virus, measles virus, mumps virus, human papillomavirus, parainfluenza virus, poliovirus, rabies virus, respiratory syncytial virus, rubella virus, and varicella-zoster virus.

In several embodiments, the population of therapeutic cells are neurons and/or neurotrophic cells. In several such embodiments, the damaged or diseased tissue is neural tissue subject to a neurodegenerative disorder. In several embodiments, the neurodegenerative disorder is selected from the group consisting of stroke, multiple sclerosis, amyotrophic lateral sclerosis, heat stroke, epilepsy, Alzheimer's disease, Parkinson's disease, Huntington's disease, dopaminergic impairment, dementia resulting from other causes such as AIDS, cerebral ischemia including focal cerebral ischemia, physical trauma such as crush or compression injury in the CNS, including a crush or compression injury of the brain, spinal cord, nerves or retina, and any other acute injury or insult producing neurodegeneration.

In several embodiments, the the magnetic field is transiently applied. In several embodiments, the magnetic field is applied via one or more magnetic sources positioned external to the damaged or diseased tissue. In some embodiments, the magnetic field is applied via a catheter having a magnetic tip. In several embodiments, the magnetic field has a field strength of about 0.1 Tesla to about 100 Tesla. In some embodiments, the magnetic field has a field strength of about 1.3 Tesla. In several embodiments, the magnetic field is applied around, adjacent to or focused in sufficient proximity to a target damaged or diseased tissue such that the magnetic composition is responsive to the magnetic field and held in a position where a therapeutic benefit can be imparted to the target tissue.

There is also provided herein a method for treating damaged or diseased cardiac tissue comprising administering to a subject having damaged or diseased cardiac tissue a composition comprising magnetic particles coupled to a first population of antibodies and a second population of antibodies, wherein the first population of antibodies is directed to a marker that expressed by population of therapeutic stem cells, wherein the population of therapeutic stem cells is endogenous to the subject, wherein the second population of antibodies is directed to a marker expressed by the damaged or diseased cardiac tissue of the subject; and applying a magnetic field around or adjacent to the damaged cardiac tissue, wherein the magnetic field enhances the targeting of the composition to the damaged or diseased cardiac tissue, wherein the magnetic field counteracts the efflux of the composition from the cardiac tissue, thereby enhancing the interaction between the second population of antibodies and the damaged or diseased cardiac tissue, thereby enhancing the delivery of the therapeutic stem cells to the damaged or diseased cardiac tissue, and wherein the enhanced delivery of the therapeutic stem cells provides long-term functional and anatomical improvements in the region of damaged cardiac tissue, thereby repairing the damaged cardiac tissue.

There is also provided herein a method for treating damaged or diseased cardiac tissue comprising administering to a subject having damaged or diseased cardiac tissue magnetic particles coupled to a first population of antibodies and a second population of antibodies, wherein the first population of antibodies is directed to the stem cell marker c-kit that is expressed by population of therapeutic stem cells, wherein the population of therapeutic stem cells is endogenous to the subject, wherein the second population of antibodies is directed to myosin light chain that is expressed by the damaged or diseased cardiac tissue of the subject; and applying a magnetic field around or adjacent to the damaged cardiac tissue, wherein the magnetic field enhances the targeting of the composition to the damaged or diseased cardiac tissue, wherein the magnetic field enhances the delivery of the composition to the damaged or diseased cardiac tissue, and wherein the enhanced delivery enables the therapeutic stem cells to repair and/or regenerate the damaged or diseased cardiac tissue.

Compositions are also provided herein. For example, in several embodiments, there is provided a composition for the targeted repair of damaged or diseased cardiac tissue comprising a magnetic particle coupled to a first population of antibodies first population of antibodies is directed against a marker expressed by a therapeutic population of cells and a second population of antibodies wherein the second population of antibodies is directed to a marker expressed by damaged or diseased cardiac tissue of the subject.

In several embodiments, the therapeutic population of cells is a population of stem cells. In several embodiments, the population of stem cells is endogenous to a subject to be treated with the composition. In several embodiments, the first population of antibodies is directed against the stem cell marker c-kit. In several embodiments, the first population of antibodies is directed against the stem cell marker CD34. In several embodiments, the first population of antibodies is directed against the stem cell marker CD45. In several embodiments, the second population of antibodies is directed against myosin light chain. In several embodiments, the composition is responsive to an applied magnetic field and as such, the application of a magnetic field enhances the delivery of the composition to the damaged or diseased cardiac tissue, wherein the enhanced delivery increases the interaction of the second population of antibodies with markers expressed by the damaged or diseased cardiac tissue, thereby increasing the delivery of the therapeutic population of cells to the damaged or diseased cardiac tissue.

In several embodiments, there is provided a method for the diagnosis of transplant rejection comprising, administering to a subject having received transplanted tissue a composition comprising, magnetic particles coupled to at least one population of antibodies directed against activated immune cells, wherein the activated immune cells comprise one or more of macrophages and T-lymphocytes, imaging the transplanted tissue of the subject to detect a signal from the magnetic particles, wherein the presence of a signal in the transplanted tissue is indicative of an immune response in the transplanted tissue, and wherein the absence of a signal in the transplanted tissue is indicative of lack of an immune response in the transplanted tissue. In several embodiments, the population of antibodies comprises one or more of antibodies directed against CD68 and antibodies directed against CD3. Depending on the embodiment, the transplanted tissue may comprise allogeneic, syngeneic, or xenogenic cells or organs.

In several embodiments, the method further comprises obtaining serum samples from the subject. In several embodiments, the serum samples are assessed for serum concentrations of one or more of protein fibrinogen (fgpro), functional fibrinogen (fgfun), C-reactive protein (CRP), and sialic acid (SA). In several embodiments, these concentrations are used to supplement the imaging based diagnosis of transplant rejection.

There are also provided herein methods for enhancing the delivery of a therapeutic population of cells to a damaged or diseased target tissue comprising administering to a subject having damaged or diseased target tissue a composition comprising magnetic particles coupled to a first population of antibodies and a second population of antibodies, wherein the first population of antibodies is directed to a marker that expressed by population of therapeutic stem cells, wherein the second population of antibodies is directed to a marker expressed by the damaged or diseased target tissue of the subject; and applying a magnetic field around or adjacent to the damaged or diseased target tissue, wherein the magnetic field increases the residence time of the composition at the damaged or diseased target tissue, thereby enhancing the interaction between the second population of antibodies and the damaged or diseased target tissue, and enhancing the delivery of the therapeutic population of cells to the damaged or diseased target tissue.

In several embodiments, the enhanced delivery the therapeutic population of cells improves the ability to image the damaged or diseased target tissue to assess the degree to which delivery is enhanced and/or to assess the therapeutic status of the the damaged or diseased target tissue. In several embodiments, the population of therapeutic cells is endogenous to the subject and is a population of immune cells. In several embodiments, the population of immune cells is delivered to a cancerous target tissue. Depending on the embodiment, the population of immune cells is selected from the group consisting of tumor-infiltrating lymphocytes, natural killer cells, cytotoxic T cells, T helper cells, T regulatory cells, antigen presenting cells, and combinations thereof.

In several embodiments, the population of immune cells is delivered to an infected target tissue, such as a tissue infected with one or more bacteria or viruses. In several embodiments, the population of immune cells is selected from the group consisting of neutrophils, monocytes, macrophages, dendritic cells, mast cells, epithelial cells, endothelial cells, fibroblasts, and mesenchymal cells. In several embodiments, the population of therapeutic cells are neurons and/or neurotrophic cells. In several embodiments, the damaged or diseased tissue is neural tissue subject to a neurodegenerative disorder.

There is also provide a method for detecting interaction between a first cell type and a second cell type, the method comprising administering to a subject a composition comprising a population of magnetic particles coupled to a first population of antibodies directed against the first cell type and a second population of antibodies directed against the second cell type, wherein the first or second cell type comprises a target tissue of interest; imaging the target tissue of the subject to detect a signal from the magnetic particles, wherein the presence of a signal in the target tissue is indicative of an interaction between the first and second cell type, and wherein the absence of a signal in the target tissue is indicative of lack of an interaction between the first and second cell type.

There is also provided a method for detecting interaction between a first cell type and a target tissue, the method comprising administering to a subject a composition comprising a population of magnetic particles coupled to a first population of antibodies directed against the first cell type and a second population of antibodies directed a marker specific to the target tissue, imaging the target tissue of the subject to detect a signal from the magnetic particles, wherein the presence of a signal in the target tissue is indicative of an interaction between the first cell type and the target tissue, and wherein the absence of a signal in the target tissue is indicative of lack of an interaction between the first cell type and the target tissue. Such embodiments are useful, for example, for assessing the degree of delivery of the first cell type to the target of interest.

There is also provide a method for detecting the presence of a magnetically-labeled therapeutic composition at a target tissue comprising imaging a target tissue of a subject having received a magnetically-labeled therapeutic composition to detect a signal from the composition, wherein the composition comprises a plurality of magnetic particle coupled to a first population of antibodies and a second population of antibodies, wherein the first population of antibodies is directed against a marker expressed a population of therapeutic cells, wherein the second population of stem cells is directed to a marker expressed by the target tissue, wherein the presence of a signal in the target tissue is indicative of an interaction between the first cell type and the target tissue, and wherein the absence of a signal in the target tissue is indicative of lack of an interaction between the first cell type and the target tissue.

Also provided is the use of a magnetic composition for the targeted repair of damaged or diseased cardiac tissue, the composition comprising a magnetic particle coupled to a first population of antibodies and a second population of antibodies, wherein the first population of antibodies is directed against a marker expressed by a therapeutic population of stem cells, and wherein the second population of stem cells is directed to a marker expressed by damaged or diseased cardiac tissue of the subject. In several embodiments, the first population of antibodies is directed against the marker CD34 and the second population of antibodies is directed against myosin light chain. In several embodiments, the therapeutic population of stem cells is endogenous to a subject to be treated with the composition,

Also provided is the use of a magnetic composition for the treatment of a target tissue afflicted with an infection due to one or more bacteria or viruses or a cancer, the composition comprising a magnetic particle coupled to a first population of antibodies and a second population of antibodies wherein the first population of antibodies is directed against a marker expressed by a therapeutic population of cells, and wherein the second population of cells is directed to a marker expressed by infected or cancerous tissue of the subject.

### BRIEF DESCRIPTION OF THE DRAWINGS

FIG. 1 depicts generally a conceptual scheme for the use of the compositions disclosed herein in the treatment of damaged cardiac tissue. In brief, the one embodiment of the methods disclosed herein involves the use of magnetic particles (shown here, for example, as an iron (Fe) particle) that are tagged with two distinct antibody populations. One antibody population is directed against a therapeutic cell population, based, for example, on identification of a marker that is present on a particular class or type of cell (shown here, for example is an anti-c-kit antibody, which will identify and “capture” stem cells expressing c-kit). Other embodiments, such as those with antibodies directed against CD34, CD45, or other therapeutic cell markers are discussed below. The second class of antibodies are directed against a target population of diseased or damaged cells (shown here for example, are anti-myosin light chain antibodies, which will target the composition to damaged cells expressing myosin light chain in a ischemic region of the heart).

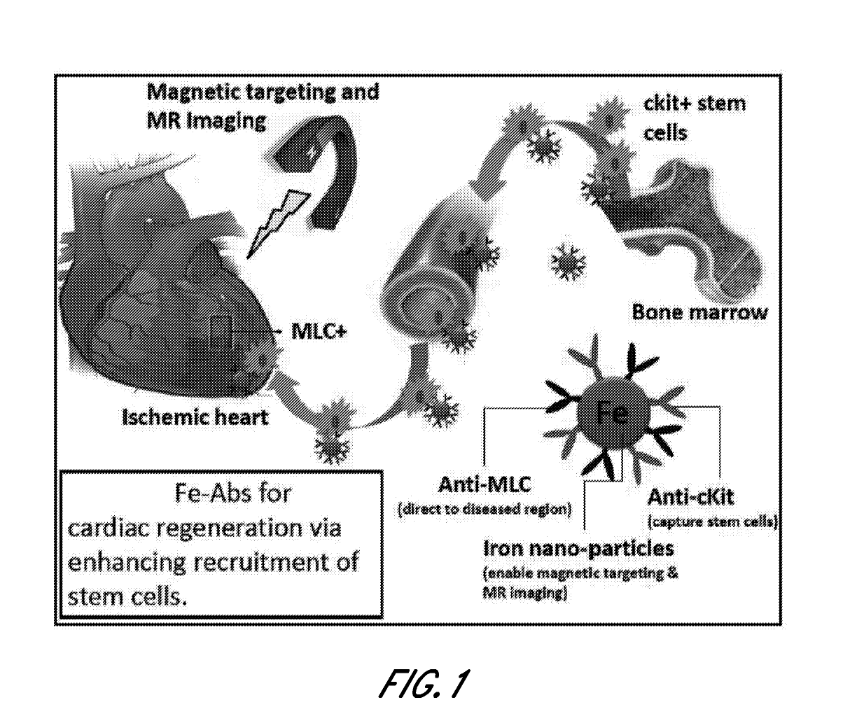

FIG. 2 depicts generally additional embodiments described herein, wherein multiple populations of magnetic particles (shown here, for example as iron particles) are conjugated with certain antibodies (in this embodiment, one type of antibody is used to label one population of magnetic particle and a second type of antibody is used to label a second population of magnetic particles.) In the embodiment shown, anti-CD3 antibodies (which will bind to lymphocytes) and anti-CD68 antibodies (which will bind to macrophages) can be administered to a subject to detect activated immune cells in a particular target tissue. The example embodiment shown depicts the detection of immune status/rejection after transplant of therapeutic stem cells to treat cardiac damage.

FIGS. 3A and 3B depicts fluorescent microscopic data indicating that iron particles can successfully be conjugated to two sets of antibodies (data depict conjugation to anti-CD45 and anti-myosin light chain (MLC) antibodies).

FIGS. 4A-4D depict additional data indicating that that iron particles can successfully be conjugated to two sets of antibodies (data depict conjugation to anti-CD45 and anti-myosin light chain (MLC) antibodies).

FIGS. 5A-5B depict data from in vitro studies that are representative of one embodiment. FIG. 5A shows neonatal rat cardiomyocytes incubated with plain iron beads (no conjugated antibodies). DAPI staining shows the nuclei of the cells, and no other fluorescent signals are detected. FIG. 5B, in contrast shows that CD45-Fe-MLC specifically binds to cardiomyocytes in vitro. Bars=10 μm.

FIGS. 6A-6B depict data that demonstrates that CD45-Fe-MLC is able to successfully link bone marrow stem cells with injured cardiomyocytes, e.g., that two populations of cells can be brought together via a bi-functional particle as described herein. Injured NRCMs were incubated with plain Fe (6A) or CD45-Fe-MLC (6B) particles and then further incubated with CD45+ bone marrow mononuclear cells (BMMNCs were engaged to injured NRCMs with CD45-Fe-MLC particles (FIG. 6B, lower right) but not with plain Fe (FIG. 6A, lower right).

FIGS. 7A-7B depict magnetic resonance imaging (MRI) detection of CD45-Fe-MLC particles after administration to rats subjected to myocardial infarction. The CD45-Fe-MLC specifically bind to the infarcted area (arrow) and the distribution of these particles could be tracked by noninvasive MRI.

FIGS. 8A-8B depict fluorescent (8A) and immunohistochemical (8B) data that confirm the existence of CD45-Fe-MLC particles in the infracted area.

FIG. 9 shows one embodiment of conjugation of antibodies to iron particles that is used in various embodiments disclosed herein.

FIGS. 10A-10B depict histological data that confirm that the experimental methods disclosed herein to replicate a myocardial infarction do in fact result in infarcted tissue (arrows).

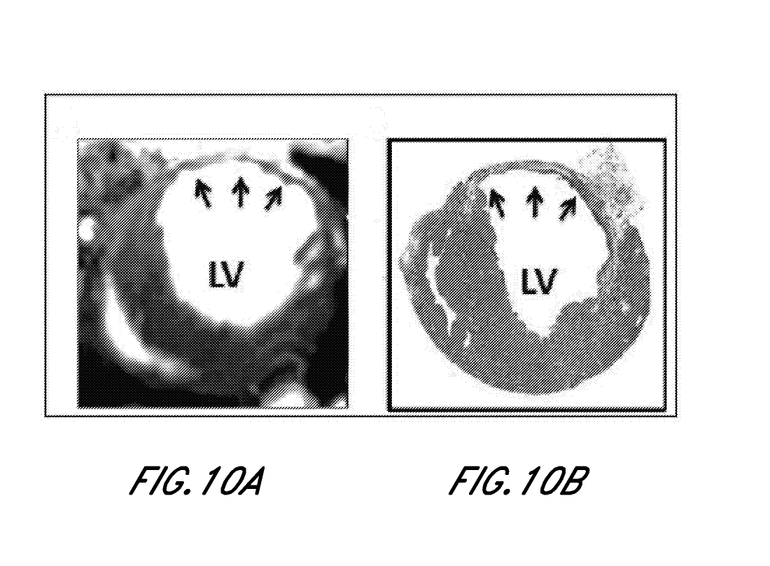

FIG. 11 depicts in vivo animal data that demonstrate that the compositions and methods disclosed herein are capable of enriching the delivery of bi-functional iron particles to a target tissue (shown as an example is the targeting of bone marrow stem cells to the liver).

FIGS. 12A-12B depict data that demonstrate that the labeling of cells directly with iron particles does not adversely impact the viability or proliferation of labeled cells.

FIGS. 13A-13C depict experimental data which demonstrate that cells captured with an antibody bound to an iron particle are magnetically responsive (13B), whereas uncaptured cells are not (13C).

FIGS. 14A-14D depict in vivo MRI data demonstrating that bone marrow stem cells can be captured by anti-CD45 antibodies coupled to an iron particle, and specifically targeted to myocardium damaged as a result of infarction. 14A shows control heart MRI data. 14B shows iron beads alone. 14C depicts targeted iron particles (arrows). 14D depicts colocalization of bi-functional iron particles and bone marrow stem cells.

### DETAILED DESCRIPTION

### General

Few families in the United States are not impacted by cardiovascular disease, which remains the leading cause of death and disability in Americans. Additionally, alone or in combination with cardiovascular disease, cancers and various types of infections affect an enormous population of individuals, both in the US and abroad. While rates of morbidity and mortality have improved, new treatments are urgently needed. Stem cell transplantation is a promising therapeutic strategy, with the notion that ex vivo-expanded cells can be used to replace/repair the diseased heart muscle. Despite the fact that numerous stem cell types are currently under investigation in clinical trials, none has been approved as a new therapy for heart disease. Salient concerns include immunogenicity, tumorigenicity, engraftment efficiency, and uncertainty regarding mechanisms of functional benefit after transplantation into the body. In the realm of cardiac injury/disease, there is strong evidence showing that when the heart is injured, endogenous stem cells are stimulated and recruited to the diseased region. Unfortunately, in some cases, this natural repair process does not suffice to offset the progressive death of cardiomyocytes after a heart attack. It is therefore desirable to develop approaches to concentrate therapeutic cells, such as stem cells, in the diseased region.

Given this need for enhanced targeting of therapeutic cells in cell therapy for treatment of damaged or diseased tissues, there is provided herein, in several embodiments, a method for treating damaged or diseased tissue comprising administering to a subject having damaged or diseased tissue a composition comprising magnetic particles coupled to a first population of antibodies and a second population of antibodies and applying a magnetic field around or adjacent to the damaged or diseased tissue, wherein the magnetic field counteracts the wash-out of the composition from the damaged or diseased tissue, thereby enhancing the delivery of the therapeutic cells to the damaged or diseased tissue, and wherein the enhanced delivery of the therapeutic cells provides therapeutic improvements in the damaged or diseased tissue, thereby treating the damaged or diseased tissue. As used herein, the terms “around” and “adjacent to” shall be given their ordinary meaning and shall also refer to generation of a magnetic field at a distance from a target tissue such magnetic particles respond to the magnetic field and are at a position sufficiently close to the target tissue (e.g., damaged or diseased tissue) to impart a therapeutically beneficial effect. Depending on the embodiment the distance may range from about 1 to about 100 millimeters, about 1 to about 20 centimeters, about 0.2 to about 5 inches, and overlapping ranges thereof. In some embodiments, the magnetic field is generated such that it is focused within less than about 10 inches, less than about 5 inches, less than about 1 inch, less than about 2 centimeters, less than about 100 millimeters, less than about 50 millimeters, less than about 10 millimeters (and overlapping ranges thereof) from the target tissue.

In several embodiments, the first population of antibodies is directed to a marker expressed by population of therapeutic cells and the second population of antibodies is directed to a marker expressed by the damaged or diseased tissue of the subject. In addition to the molecular targeting provided by the second population of antibodies, in several embodiments, the magnetic field counteracts the wash-out of the composition from the damaged or diseased tissue and further enhances the molecular targeting of the composition via the enhanced interaction between the second population of antibodies and the markers expressed by the damaged or diseased tissue. In several embodiments, therefore, the combination of molecular and magnetic targeting act synergistically to improve retention of the composition at the target site, which leads to unexpectedly beneficial therapeutic improvements of the target tissue.

Advantageously, the methods and compositions disclosed herein are flexible in that they can be applied to provide a therapeutic effect to a wide variety of damaged or diseased tissues, using a wide variety of therapeutic cell types. For example, in several embodiments the damaged or diseased tissue comprises infected tissue, such as tissue infected with one or more of bacteria, fungi, viruses, parasites or combinations thereof. Infections may be short term (e.g., acute episodes), chronic infections, or recurrent infections. Infections caused by primary and/or opportunistic pathogens are also treated in several embodiments. In several embodiments, the therapeutic improvements comprise one or more of the inhibition, removal, or elimination of the infectious agent. In some embodiments, the inhibition of the agent using the methods and compositions disclosed herein is used in conjunction with traditional anti-infection therapies (e.g., topical or oral anti-biotics, anti-virals etc).

In several embodiments, the therapeutic improvements comprise functional or anatomical repair of the damaged or diseased tissue. For example, a particular disease may result in necrosis, apoptosis, or other loss of cells/tissue. In some embodiments, the compositions and methods provided herein target a population of therapeutic cells that can repair and/or regenerate the lost cells/tissues. In some embodiments, functional repair of the tissue results (either in addition to or in place of anatomical repair). In some embodiments, damage to tissue is caused by injury (e.g., trauma or other adverse event, such as an ischemic episode, etc.) and is treated with the method and compositions herein through functional and/or anatomical improvement of the damaged tissue. As a non-limiting example, if a muscular tissue is damaged due to a period of lack of blood flow, in some embodiments, the compositions, when molecularly and/or magnetically targeted to the damaged tissue serve to supplement (e.g., improve) the function of the existing cells of the tissue, and/or in some embodiments, led to the generation of new replacement cells.

In several embodiments, the population of therapeutic cells is endogenous to the subject. This provides several advantages in certain embodiments, namely, i) there is no requirement for administration of cells because the population therapeutic cells already exists within the subject to be treated, ii) the lack of a need for exogenous cells eliminates the chances of rejection of the therapeutic cells (e.g., as non-self cells), and iii) the lack of a need for exogenous cells reduces the risk of complications (such as infections) because there is no requirement for in vitro growth and/or manipulation of the population of therapeutic cells prior to use in the methods disclosed herein.

Although endogenous cell therapy has several advantages, in several embodiments, the population of therapeutic cells is exogenous to the subject. Exogenous cells also present certain advantages, such as the ability to control precisely the dose of therapeutic cells, the ability to select a population of therapeutic cells that may be limited or non-existent in a certain subject, the ability to genetically manipulate the therapeutic cells and/or modify the interaction between the cells and the antibodies on the magnetic particles (e.g., to generate a stronger interaction, and/or the ability to administer a customized mixture of various therapeutic cell types in combination.

In several embodiments, the magnetic particles comprise superparamagnetic iron oxides (SPIO). In some embodiments, the magnetic particles have a diameter of about 10 to about 10,000 nanometers. In some embodiments, the magnetic particles are covalently coupled to the first and second populations of antibodies. In several embodiments, the covalent coupling is achieved by modification of carboxyl groups coating (or otherwise attached to) the magnetic particles. Other types of coupling are used in other embodiments, such as for example, electrostatic coupling, secondary antibody-antigen interactions (e.g., biotin-streptavidin), or other physical couplings such as protein-protein interactions or antibodies that are impregnated into a coating placed around the microparticle. Depending on the embodiment, the magnetic particles after coupling to the antibodies have a diameter of about 30 to 15000 nanometers.

Specific markers are used in several embodiments to target particular populations of therapeutic cells and/or specifically identifiable damaged or diseased target tissues. For example, in several embodiments the first population of antibodies is directed to the c-kit marker expressed on stem cells. In several embodiments, the first population of antibodies recognizes CD34. In several embodiments, the first population of antibodies recognizes CD45 on stem cells. In some embodiments, the stem cells are bone marrow stem cells. Other markers are recognized in other embodiments, depending on the type of therapeutic cells employed in the various embodiments. In several embodiments, the damaged or diseased tissue comprises damaged or diseased cardiac tissue and the second population of antibodies is directed to a marker expressed only (or preferentially) by damaged or diseased cardiac tissue. In several embodiments, the marker is myosin light chain. In some embodiments, the cardiac tissue has been damaged by an acute adverse cardiac event such as, for example, a myocardial infarction. In other embodiments, the damaged cardiac tissue results from chronic stress or disease of the heart such as, for example, chronic heart failure, systemic hypertension, pulmonary hypertension, valve dysfunction, congestive heart failure, coronary artery disease, and combinations thereof.

In several embodiments, functional improvement of damaged or diseased cardiac tissue comprises an increase in cardiac output and/or an increase in left ventricular ejection fraction (of greater than 2%, 5%, 10% or more). In some embodiments, anatomical improvements in the damaged or diseased cardiac tissue comprise an increase in viable cardiac tissue, an increase in cardiac wall thickness, and/or a decrease in scar tissue formation.

Other damaged or diseased tissues are targeted in other embodiments, including but not limited to cancerous cells, infected cells, necrotic cells, apoptotic cells, cells subjected to and damaged from physical trauma, foreign bodies, non-self cells leading to immune rejection, and the like.

Moreover, to facilitate a variety of treatments, there is also provided for herein a composition for the targeted repair of damaged or diseased tissue comprising a magnetic particle coupled to a first population of antibodies and a second population of antibodies, wherein the first population of antibodies is directed against a marker expressed by a therapeutic population of stem cells, wherein the therapeutic population of stem cells is endogenous to a subject to be treated with the composition, wherein the second population of stem cells is directed to a marker expressed by damaged or diseased tissue of the subject.

In some embodiments, the damaged or diseased tissue comprises damaged or diseased cardiac tissue. In some such embodiments, the first population of antibodies of the composition is directed against the stem cell marker c-kit. In some embodiments, the second population of antibodies is directed against myosin light chain.

In several embodiments, the composition is responsive to an applied magnetic field, which in several embodiments, enhances the delivery of the composition to the damaged or diseased tissue, wherein the enhanced delivery increases the interaction of the second population of antibodies with markers expressed by the damaged or diseased tissue, thereby increasing the delivery of the therapeutic population of cells to the damaged or diseased tissue.

In several embodiments, the magnetic field is transiently applied at or around a target tissue. The location and depth of the target tissue will, at least in part, define the mode by which the magnetic field is applied. For example, a different magnetic field strength and/or focus may be required for a deep organ such as the liver or intestine, as compared to the magnetic field required for a surface organ such as skin or skeletal muscle. In some embodiments, a single magnetic source is used, whereas in other embodiments, multiple sources are used and the resultant field is defined by the area of interaction between the field (e.g., the fields “triangulate” to generate a certain magnetic field strength in the target region). For example, in several embodiments, the magnetic field is applied via one or more magnetic sources positioned external to the heart. In other embodiments, internal magnetic sources are used, such as, for example, delivery catheter having a magnetic tip. In several embodiments, the magnetic field has a field strength of about 0.1 Tesla to about 100 Tesla. In some embodiments, the magnetic field has a field strength of about 1.3 Tesla.

Delivery of the compositions can be by various routes, depending on the embodiment. For example, in several embodiments, the composition is delivered systemically. In such embodiments, the molecular and magnetic targeting serve to enrich the composition at the target tissue. In some embodiments, the route of systemic administration is selected from the group consisting of intravenous, intra-arterial, intracoronary, and intraventricular administration.

Given the high rate of cardiovascular disease in modern societies, there is also provided herein, in several embodiments, a method for treating damaged or diseased cardiac tissue comprising administering to a subject having damaged or diseased cardiac tissue a composition comprising magnetic particles coupled to a first population of antibodies and a second population of antibodies, wherein the first population of antibodies is directed to a marker that expressed by population of therapeutic stem cells, wherein the population of therapeutic stem cells is endogenous to the subject, wherein the second population of antibodies is directed to a marker expressed by the damaged or diseased cardiac tissue of the subject; and applying a magnetic field around or adjacent to the damaged cardiac tissue, wherein the magnetic field enhances the targeting of the composition to the damaged or diseased cardiac tissue, wherein the magnetic field counteracts the efflux of the composition from the cardiac tissue, thereby enhancing the interaction between the second population of antibodies and the damaged or diseased cardiac tissue, thereby enhancing the delivery of the therapeutic stem cells to the damaged or diseased cardiac tissue, and wherein the enhanced delivery of the therapeutic stem cells provides long-term functional and anatomical improvements in the region of damaged cardiac tissue, thereby repairing the damaged cardiac tissue.

There is also provided a method, in one embodiment, for treating damaged or diseased cardiac tissue comprising administering to a subject having damaged or diseased cardiac tissue a composition comprising magnetic particles coupled to a first population of antibodies and a second population of antibodies, wherein the first population of antibodies is directed to the stem cell marker c-kit that is expressed by population of therapeutic stem cells, wherein the population of therapeutic stem cells is endogenous to the subject, wherein the second population of antibodies is directed to myosin light chain that is expressed by the damaged or diseased cardiac tissue of the subject; and applying a magnetic field around or adjacent to the damaged cardiac tissue, wherein the magnetic field enhances the targeting of the composition to the damaged or diseased cardiac tissue, wherein the magnetic field enhances the delivery of the composition to the damaged or diseased cardiac tissue, and wherein the enhanced delivery enables the therapeutic stem cells to repair and/or regenerate the damaged or diseased cardiac tissue. In some embodiments, rather than a first population of antibodies that is directed to c-kit, the first population is directed to CD34. In some embodiments, the first population is directed to CD45. In still additional embodiments, a mixed first population of antibodies is employed, thereby enhancing delivery of one or more therapeutic cell types.

In several embodiments, there is provided a minimally-invasive method for the diagnosis of transplant rejection comprising: administering to a subject having received transplanted tissue a composition comprising, magnetic particles coupled to at least one population of antibodies directed against activated immune cells, wherein the activated immune cells comprise one or more of macrophages and T-lymphocytes, imaging the transplanted tissue of the subject to detect a signal from the magnetic particles, wherein the presence of a signal in the transplanted tissue is indicative of an immune response in the transplanted tissue, and wherein the absence of a signal in the transplanted tissue is indicative of lack of an immune response in the transplanted tissue.

In several embodiments, the transplanted tissue comprises allogeneic, syngeneic, or xenogenic cells or organs. In several embodiments, the administration is via a systemic delivery route selected from the group consisting of intravenous, intra-arterial, intracoronary, and intraventricular administration. In several embodiments, the population of antibodies comprises one or more of antibodies directed against CD68 and antibodies directed against CD3, or a combination thereof. In some embodiments, as discussed above, the magnetic particles comprise superparamagnetic iron oxides (SPIO), such as for example, monodispered SPIO.

In some embodiments, the method further comprises obtaining serum samples from the subject and measuring serum concentrations of one or more of protein fibrinogen (fgpro), functional fibrinogen (fgfun), C-reactive protein (CRP), and sialic acid (SA) to supplement the imaging based diagnosis of transplant rejection.

In several embodiments, there is provided a method for enhancing the delivery of a therapeutic population of cells to a damaged or diseased target tissue comprising administering to a subject having damaged or diseased target tissue a composition comprising magnetic particles coupled to a first population of antibodies and a second population of antibodies, wherein the first population of antibodies is directed to a marker that expressed by population of therapeutic stem cells, wherein the second population of antibodies is directed to a marker expressed by the damaged or diseased target tissue of the subject; and applying a magnetic field around or adjacent to the damaged or diseased target tissue, wherein the magnetic field increases the residence time of the composition at the damaged or diseased target tissue, thereby enhancing the interaction between the second population of antibodies and the damaged or diseased target tissue, and enhancing the delivery of the therapeutic population of cells to the damaged or diseased target tissue.

In several embodiments, the enhanced delivery the therapeutic population of cells improves the ability to image the damaged or diseased target tissue to assess the degree to which delivery is enhanced and/or to assess the therapeutic status of the damaged or diseased target tissue. In some embodiments, the population of therapeutic cells is endogenous to said subject while in other embodiments, they are exogenous. In several embodiments, the said population of therapeutic cells a population of immune cells. In several embodiments, said population of immune cells is selected from the group consisting of tumor-infiltrating lymphocytes, natural killer cells, cytotoxic T cells, T helper cells, T regulatory cells, and antigen presenting cells. In some embodiments, the population of immune cells is delivered to a cancerous target tissue.

In several embodiments, the cancerous target tissue is affected with one or more cancers selected from the group consisting of acute Lymphoblastic Leukemia (ALL), Acute Myeloid Leukemia (AML), Adrenocortical Carcinoma, Kaposi Sarcoma, Lymphoma, gastrointestinal cancer, appendix Cancer, Central Nervous System cancer, basal Cell Carcinoma, Bile Duct Cancer, Bladder Cancer, Bone Cancer, Brain Tumors (including but not limited to Astrocytomas, Spinal Cord Tumors, Brain Stem Glioma, Craniopharyngioma, Ependymoblastoma, Ependymoma, Medulloblastoma, Medulloepithelioma, Breast Cancer, Bronchial Tumors, Burkitt Lymphoma, cervical cancer, colon cancer, Chronic Lymphocytic Leukemia (CLL), Chronic Myelogenous Leukemia (CIVIL), Chronic Myeloproliferative Disorders, ductal carcinoma, endometrial cancer, esophageal cancer, gastric cancer, hodgkin lymphoma, hairy cell leukemia, renal cell cancer, leukemia, oral cancer, liver cancer, lung cancer, lymphoma, melanoma, ocular cancer, ovarian cancer, pancreatic cancer, prostate cancer, pituitary cancer, uterine cancer, and vaginal cancer.

Alternatively, in several embodiments, the population of immune cells is delivered to an infected target tissue, such as a target tissue is infected with one or more bacteria, viruses, fungi, and/or parasites. In such embodiments, the population of immune cells is selected from the group consisting of neutrophils, monocytes, macrophages, dendritic cells, mast cells, epithelial cells, endothelial cells, fibroblasts, and mesenchymal cells. In some embodiments, the infection is bacterial in origin and the infectious bacteria is selected the group of genera consisting of Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, and Yersinia, and mutants or combinations thereof.

In some embodiments, the infection is viral in origin and the result of one or more viruses selected from the group consisting of adenovirus, Coxsackievirus, Epstein-Barr virus, hepatitis a virus, hepatitis b virus, hepatitis c virus, herpes simplex virus, type 1, herpes simplex virus, type 2, cytomegalovirus, ebola virus, human herpesvirus, type 8, HIV, influenza virus, measles virus, mumps virus, human papillomavirus, parainfluenza virus, poliovirus, rabies virus, respiratory syncytial virus, rubella virus, and varicella-zoster virus.

In still additional embodiments, the population of therapeutic cells are neurons and/or neurotrophic cells. In such embodiments, the damaged or diseased tissue is neural tissue subject to a neurodegenerative disorder. In several embodiments, the neurodegenerative disorder is selected from the group consisting of stroke, multiple sclerosis, amyotrophic lateral sclerosis, heat stroke, epilepsy, Alzheimer's disease, Parkinson's disease, Huntington's disease, dopaminergic impairment, dementia resulting from other causes such as AIDS, cerebral ischemia including focal cerebral ischemia, physical trauma such as crush or compression injury in the CNS, including a crush or compression injury of the brain, spinal cord, nerves or retina, and any other acute injury or insult producing neurodegeneration.

In several embodiments, there are provided methods for detecting interaction between multiple cell types, the method comprising administering to a subject a composition comprising a population of magnetic particles coupled to a first population of antibodies directed against said first cell type and a second population of antibodies directed against said second cell type, and imaging a region of interest in of said subject to detect a signal from said magnetic particles. In some embodiments, the methods allow the detection of colocalization of the multiple cell types (e.g., by increased signal detection). In some embodiments, the detection of interaction is quantitative, while in other embodiments it is binary (e.g., indicating only the presence or absence of some interaction).

There is also provided a method for detecting interaction between a first cell type and a target tissue, the method comprising administering to a subject a composition comprising a population of magnetic particles coupled to a first population of antibodies directed against said first cell type and a second population of antibodies directed a marker specific to said target tissue, and imaging said target tissue of said subject to detect a signal from said magnetic particles, wherein the presence of a signal in said target tissue is indicative of an interaction between said first cell type and said target tissue, and wherein the absence of a signal in said target tissue is indicative of lack of an interaction between said first cell type and said target tissue.

In additional embodiments, there is provided a method for detecting interaction between a first cell type and a target tissue, the method comprising administering to a subject a composition comprising a population of magnetic particles coupled to a first population of antibodies directed against said first cell type and a second population of antibodies directed a marker specific to said target tissue, imaging said target tissue of said subject to detect a signal from said magnetic particles, wherein the presence of a signal in said target tissue is indicative of an interaction between said first cell type and said target tissue, and wherein the absence of a signal in said target tissue is indicative of lack of an interaction between said first cell type and said target tissue. As discussed herein, the first cell type can be any variety of cell, including a cell administered (or recruited from endogenous stores) for therapeutic purposes, an immune cell (e.g., lymphocytes, anti-inflammatory cells, etc), or a stem cell. In some embodiments, the target tissue is a tissue that has or is affected by damage, disease, infection and the like (e.g., cancer, ischemia, infection, physical trauma, neurodegeneration etc.). Thus, in several embodiments the methods provided for herein are advantageous in that they not only assist in the targeting of a therapeutic to a target tissue, but allow, via imaging, the confirmation that the therapeutic has arrived at the target tissue.

In several additional embodiments, there are provided methods for detecting the presence of a magnetically-labeled therapeutic composition at a target tissue comprising imaging a target tissue of a subject having received a magnetically-labeled therapeutic composition to detect a signal from said composition, wherein the presence of a signal in said target tissue is indicative of an interaction between said first cell type and said target tissue, and wherein the absence of a signal in said target tissue is indicative of lack of an interaction between said first cell type and said target tissue. In several embodiments, the composition comprises a plurality of magnetic particle coupled to a first population of antibodies and a second population of antibodies, wherein said first population of antibodies is directed against a marker expressed a population of therapeutic cells, wherein said second population of stem cells is directed to a marker expressed by said target tissue. In several embodiments, this approach allows the determination of whether (and in some embodiments, what quantity) of a magnetically-labeled therapeutic composition has been deployed to a target tissue. In some embodiments, the imaging modality (e.g., magnetic resonance imaging) not only enables the imaging, but in certain embodiments, further enhances the targeting of the magnetically-labeled therapeutic composition. Thus, in several embodiments the diagnostic assessment of the presence of a magnetically-labeled therapeutic composition itself further improves the delivery of the composition.

Prior experiments have demonstrated that magnetic targeting of ex vivo-expanded stem cells labeled with iron particles and then delivered to the heart under external magnetic field increased cell engraftment rates by 3-fold and therapeutic functional benefit was achieved. Additional experiments have demonstrated molecular targeting of stem cells armed with bi-functional antibodies (e.g., CD45 and MLC; tagged in vitro) followed by systemic delivery, which led to accumulation of the cells in the ischemic heart (expressing MLC) augmentation of cardiac function. Both of the above-described approaches require transplantation of exogenous stem cells and are directed to enhancing the effects of those exogenous cells. In several embodiments, the compositions and methods disclosed herein advantageously allow the functional augmentation and/or repair of damaged or diseased tissues (e.g., cardiac tissue) without the requirement for exogenous cells. This approach combines both molecular and magnetic targeting in such a way that synergy between the two modalities is achieved, in some embodiments, thereby leading to a more robust repair of damaged tissue and/or improved function. In several embodiments, a magnetic particle is coupled to two antibodies. In some embodiments, a first antibody linked to the particle can be used to link the particle to a particular therapeutic cell type, for example a cardiac stem cell, based on known antigens expressed on the surface of that cell type. In some embodiments, a first antibody directed to c-kit can be used to selectively enrich a c-kit positive population of stem cells. In some embodiments, a first antibody directed to CD34 is used to selectively enrich a CD34 positive population of stem cells. In some embodiments, a first antibody directed to CD45 can be used to selectively enrich a CD45 positive population of stem cells. Other markers can also be used, depending on the embodiment. In some embodiments, antibodies can be directed against an antigen that is genetically manipulated. For example, a non-native antigen may be engineered to be expressed on a therapeutic cell, for example a liver-specific marker on a non-liver cell type. In this manner, a particular population of cells known to be genetically modified may be selectively enriched. A second antibody linked to the magnetic particle is directed to a known antigen on a desired target tissue. For example, in some embodiments, the second antibody recognizes a cardiac tissue specific marker. Thus, molecular and magnetic targeting in combination, in some embodiments, are used to accomplish one or more of: selective enrichment of therapeutic cells, selective targeting of specific target cells based on antigen expression, and magnetic enhancement of therapeutic cell retention at a specific target tissue. In some embodiments, the efficiency of the therapeutic cell delivery is enhanced, by way of improvements in the delivery, retention, and/or engraftment of the cells bound to the particle via the antibody (or antibodies). As discussed in more detail below, the magnetic particles are also useful, in several embodiments, for enhanced imaging procedures, though in some embodiments, the magnetic particles are not used for imaging or visualization purposes.

In addition to the methods of treatment and repair described herein, several embodiments also allow for the noninvasive imaging of inflammation and immune reactions in various organs that have recently received transplanted cells. Detection of immune reactions in the heart in the most accurate manner possible is a challenge that those of skill in the art are still working to overcome. Acute rejection is traditionally diagnosed by endomyocardial biopsy, which, in some cases, is prone to sampling error because of the limited sizes and locations of tissue available, particularly in pediatric patients. More importantly, discrepancies between biopsy-based diagnosis and actual rejection may be found. Naked iron nanoparticles (as contrast agents) and MRI have been used for noninvasive detection of acute cardiac allograft rejection have provided intriguing preliminary results, but this approach heavily depends on the efficiency of endocytosis of injected iron particles by macrophages. However, T lymphocytes, a major player in acute immune rejection, are not prone to endocytose iron particles. Thus, in some embodiments, bifunctional particles that capture T lymphocytes and detect macrophages create a specific MRI signal diagnostic for rejection.

In several embodiments, to enhance endogenous stem cell recruitment, bi-functional compositions (also referred to herein as Fe-Abs), with 2 types of antibodies (Ab1 binds to a specific stem cell population and Ab2 binds to the diseased tissue). When delivered into the body, this agent will capture mobilized stem cells (expressing Ab1) and then direct them to the injured heart muscle (expressing Ab2). Advantageously, for both the therapeutic aspects and the diagnostic aspects, magnetic particles provide the ability to use magnetic targeting to physically enrich the cells (for example, stem cells) in the heart (or other target organ) and MRI can be used to monitor the injected particles and/or noninvasive detection of immune rejection, anti-immune cell (e.g. T lymphocytes, macrophages). In particular, cardiac cells may be delivered to damaged cardiac tissue and the enhanced delivery, retention and/or the engraftment of the cells facilitates repair and/or regeneration of cardiac tissue. In some embodiments, in addition to the application of magnetic field, the bi-functional compositions are optionally administered in combination with one or more vascular permeability agents. Further information regarding administration of vascular permeability agents can be found in International Patent Application No. PCT/US10/54358, filed, Oct. 27, 2010, which is incorporated in its entirety by reference herein.

The compositions and methods provided herein are useful for facilitating delivery of cells to target tissues or organs and improving the retention rate and/or engraftment of administered cells. In several embodiments, the compositions and methods provided herein are useful for capturing and targeting endogenous cells, for example endogenous stem cells, to damaged cardiac tissue and improving on the typically low retention rate of the delivered cells (largely due to the wash-out effect caused by blood flow and the contraction of the heart). In general cardiac retention rate of delivered cells (by a variety of typical routes such as intramyocardial, intracoronary, intravenous) ranges from about 11% to less than 1% retention.

As such, the compositions and methods provided herein can be used to treat a variety of heart diseases or disorders. The compositions and methods provided herein can also be used for the treatment of other diseases or disorders, for example, hepatic diseases, cancers, neurodegenerative diseases or diabetes, and diseases involving digestive and urogenital systems, among others.

Moreover, in several embodiments, the compositions and methods herein are useful as a diagnostic tool. For example, as disclosed in more detail below, certain antibodies can be linked to magnetic particles such that the antibodies will target tissues undergoing an immune reaction, such as tissues that have been recently transplanted (or treated with cell therapy) and may be undergoing an immune rejection. The delivery of the magnetic particles to such tissues subsequently enables the non-invasive imaging of the tissues (e.g., by MRI) to assess their rejection status. As used herein, the term “non-invasive” shall be given its ordinary meaning and shall also be used interchangeably with “minimally invasive” and shall also refer to methods and procedures disclosed herein wherein an injection or infusion of bi-functional compositions (as disclosed herein) are administered, but no additional invasive steps are performed.

### Types of Therapeutic Cells

Cells useful in the compositions and methods provided herein include any type of cells known in the art expressing a particular marker, either naturally or due to genetic modification. The presence of a marker, which in some embodiments is unique to a particular desired cell population, allows the selective “capture” of those cells by the bifunctional compositions disclosed herein. Cells used in the methods and compositions disclosed herein can be obtained or derived from any of a variety of sources. For example, subjects that can be the donors (or recipients) of stem cells in the methods presented herein include, for example, mammals, such as non-primates (e.g., cows, pigs, horses, cats, dogs; rats or rabbits) or primates (e.g., monkeys or humans). In some embodiments, the subject is a human. In one embodiment, the subject is a mammal, e.g., a human, such as a human with acute or chronic heart failure or other cardiac tissue injury, cancer, or an infection.

While a single species or individual can be the donor by providing the cells and be the recipient by receiving the cells (i.e., autologous stem cells), in some embodiments the donor and recipient of the stem cells may be of different species (i.e., xenogeneic). For instance, porcine cells can be administered into human cardiac tissue. In some embodiments, the stem cells are allogeneic or syngeneic. In some embodiments, the stem cells are autologous to the cardiac tissue. Having an autologous source of stem cells from the same individual further decreases the possibility of avoiding transplant rejection such as Graft-versus-Host Disease (GVHD). In some embodiments, the autologous stem cells are derived from adult non-cardiac tissue. In some embodiments, the stem cells are induced pluripotent stem cells derived or created from somatic adult cells, e.g., dermal fibroblasts, using techniques known in the art.

### Stem Cells

In some embodiments, stem cells are preferred as a therapeutic cell type, for example for their pluripotency and the resultant ability to generate a wide variety of tissue types. As used herein, the term “stem cells” shall be given its ordinary meaning and shall also refer to cells that have the capacity to self-renew and to generate differentiated progeny. The term “pluripotent stem cells” shall be given its ordinary meaning and shall also refer to stem cells that can give rise to cells of all three germ layers (endoderm, mesoderm and ectoderm), but do not have the capacity to give rise to a complete organism. The term “multipotent stem cells” shall be given its ordinary meaning and shall also refer to a stem cell that has the capacity to grow into a subset of the fetal or adult mammalian body's approximately 260 cell types. For example, some multipotent stem cells can differentiate into at least one cell type of ectoderm, mesoderm and endoderm germ layers. The term “progenitor cell” shall be given its ordinary meaning and shall also refer to a cell that has the ability to self-renew, generally for a limited number of times, and can also give rise to a particular cell type or limited group of cell types. The term “bone marrow stem cells” shall be given its ordinary meaning and shall also refer to stem cells obtained from or derived from bone marrow. In several embodiments, stem cells useful for the compositions and methods provided herein include, mesenchymal stem cells (such as bone marrow or adipose stem cells), endothelial progenitor cells, cardiac stem cells, hepatic stem cells, pancreatic stem cells, hematopoietic stem cells, muscle stem cells (e.g., myocyte progenitor cells), epithelial stem cells, vascular stem cells, and other stem cell types commonly known in the art. As several embodiments disclosed herein enable the recruitment and targeting of endogenous stem cells from a subject, the cells are necessarily autologous.

However, in some embodiments, (e.g., in an immune compromised subject or a subject having low counts of endogenous therapeutic cells), exogenous cells may also be used. The cells employed can be autologous or heterologous to the subject being treated. In such embodiments, the stem cells that can be used include, but are not limited to embryonic, adult stem cells, amniotic stem cells, bone marrow stem cells, placenta-derived stem cells, embryonic germ cells, cardiac stem cells, cardiospheres, cardiosphere-derived cells, induced pluripotent stem cells, mesenchymal stem cells, endothelial progenitor cells, adipose-derived stem cells, cord placenta-derived stem cells, embryonic germ cells, induced pluripotent stem cells, cord blood stem cells, spermatocytes and other cell types known in the art which can be obtained from a subject, cultured (e.g., expanded) and introduced into a subject (either the same or second subject). Additional embodiments employ adult cells of any variety (either those harvested from a subject and re-administered, or those harvested from a donor). As used herein, the term “embryonic stem cells” shall be given its ordinary meaning and shall also refer to stem cells derived from the inner cell mass of an early stage embryo, e.g., human, that can proliferate in vitro in an undifferentiated state and are pluripotent. The term “placenta-derived stem cells” or “placental stem cells” shall be given their ordinary meanings and shall also refer to stem cells obtained from or derived from a mammalian placenta, or a portion thereof (e.g., amnion or chorion). The term “amniotic stem cells” shall be given its ordinary meaning and shall also refer to stem cells collected from amniotic fluid or amniotic membrane. The term “embryonic germ cells” shall be given its ordinary meaning and shall also refer to cells derived from primordial germ cells, which exhibit an embryonic pluripotent cell phenotype. The term “spermatocytes” shall be given its ordinary meaning and shall also refer to male gametocytes derived from a spermatogonium.

In several embodiments, the compositions and methods herein are used to treat neurodegenerative disorders. Antibodies specific to neural progenitor cells are used, in several embodiments, to capture the progenitor cells and (either with or without magnetic targeting) deliver the cells to sites of neural injury or degeneration. As used herein, the terms “neurodegeneration” and “neurodegenerative disorders” shall be given their ordinary meanings and shall also refer cell destruction resulting from destructive events such as stroke or trauma as well as delayed or progressive destructive mechanisms that are invoked by cells due to the occurrence of such destructive events. Destructive events include disease processes or physical injury or insult, multiple sclerosis, amyotrophic lateral sclerosis, heat stroke, epilepsy, Alzheimer's disease, Parkinson's disease, Huntington's disease, dopaminergic impairment, dementia resulting from other causes such as AIDS, cerebral ischemia including focal cerebral ischemia, and physical trauma such as crush or compression injury in the CNS, including a crush or compression injury of the brain, spinal cord, nerves or retina, or any other acute injury or insult producing neurodegeneration.

Depending on the embodiment and the capturing antibody employed, the captured stem cells can be a homogeneous population (e.g., all bone marrow cells) while in some embodiments the stem cells can be a mixed cell population (e.g., a mixture of bone marrow and cardiac stem cells). In still additional embodiments, the population can be enriched with a particular type of stem cell. Homogeneous cell compositions can be obtained, for example, by recognizing (and selecting for cells with) cell surface markers characteristic of stem cells, or particular types of stem cells (or other desired type of therapeutic cell), in conjunction with monoclonal antibodies directed to the specific cell surface markers. Homogenous cell compositions, for example, those comprising CDCs, can also be obtained without the use of antibody reagents for selection using standard techniques (see, e.g., Smith et al. (2007) Circulation 115:896), which is incorporated by reference herein.

In the event that exogenous cells are used (for example, as a supplemental or pre-treatment therapy), the exogenous cells may also be autologous, syngeneic, xenogeneic, or allogeneic to the subject being treated. Exogenous cells, when used in certain embodiments, may include stem cells (or other types of therapeutic cell) that are specific for treating a particular target tissue (for example, cardiac-derived therapeutic cells, such as cardiospheres or cardiosphere-derived cells (CDCs). Additional information regarding cardiospheres, CDCs and various applications thereof may be found in, for example, U.S. Pat. No. 8,268,619, issued Sep. 28, 2012, U.S. patent application Ser. No. 11/666,685, filed Apr. 21, 2008 and International Patent Application No. PCT/US10/54358, filed, Oct. 27, 2010, each of which is incorporated in its entirety by reference herein.

### Non-Stem Cells

Certain cell therapies do not rely upon stemness of administered cells, but rather upon another desirable feature of targeting cells to any given tissue. In some embodiments, cell types other than stem cells (e.g., adult or partially differentiated cells) are used as the therapeutic cells that are captured and delivered to a target tissue per the compositions and methods provided herein. Choice of a particular cell type can be determined by the particular tissue or organ for which delivery or treatment is desired. For example, in several embodiments immune cells that may kill or inhibit tumors are targeted to cancerous tissues, while in another embodiment phagocytic cells are targeted to sites of infection such as abscesses. In some embodiments, for example, the bone marrow is stimulated to release certain progenitor cells, which begin the maturation process, and can be captured by the bifunctional compositions disclosed herein and directed to a target tissue for therapy. In several embodiments, circulating cells are preferred as the therapeutic cells, as access to such cells in the blood stream is more easily accomplished. In other embodiments, however, non-circulating cells can be used as the therapeutic cells. In certain such embodiments, an exogenous liberation step (e.g., stimulation of bone marrow) is performed in advance to move the cells to the blood stream. In other embodiments, however, such a step is not necessary, as the damage to a target organ provides a natural signaling cascade that liberates the cells, allowing the compositions disclosed herein to access the target cell population. In some embodiments, cardiac cells, endothelial cells, fibroblasts and/or smooth muscle cells make up at least a portion of the therapeutic cell population (e.g., for treatment of cardiac damage). In some embodiments, endothelial cells, fibroblasts and/or hepatocytes make up at least a portion of the therapeutic cell population (e.g., for treatment of hepatic damage). In one embodiment, neural cells, neuroglial cells, endothelial cells and/or fibroblasts make up at least a portion of the therapeutic cell population (e.g., for treatment of neural or spinal cord damage). In one embodiment, endothelial cells, fibroblasts, pancreatic islet cells and/or other pancreatic cells make up at least a portion of the therapeutic cell population (e.g., for treatment of pancreatic damage). In some embodiments, fibroblasts and/or respiratory epithelial cells make up at least a portion of the therapeutic cell population (e.g., for treatment of lung or respiratory damage). In one embodiment, endothelial cells, smooth muscle cells and/or fibroblasts make up at least a portion of the therapeutic cell population (e.g., for treatment of vessel damage and/or atherosclerosis). In another embodiment, endothelial cells, epithelial cells, fibroblasts and/or smooth muscle cells make up at least a portion of the therapeutic cell population (e.g., for treatment of gastrointestinal or urogenital tissues).

In several embodiments, cells that target cancerous cells are used (e.g., captured by a first population of magnetically coupled antibodies). In some embodiments, immune cells (e.g. cytotoxic T cells) can be captured by bifunctional compositions disclosed herein and directed to targeted cancer cells and kill, inhibit, or otherwise interfere with the cancer cells. For example, an FDA-approved T cell therapy (PROVENGE®) utilizes antigen presenting cells to activate the body's own T cells to attack prostate cancerous tissues. In several embodiments, bifunctional compositions as disclosed herein provide synergistic enhancement of the therapeutic outcomes by directing (including magnetically, molecularly, or both) the PROVENGE®-activated T cells to the prostate cancer cells. In several embodiments, tumor-infiltrating lymphocytes are captured for use as the therapeutic cell population, in addition to, optionally, natural killer cells, cytotoxic T cells, T helper cells, T regulatory cells, antigen presenting cells, and the like. In several embodiments, a single type of lymphocyte is captured, while in other embodiments, more than one type is captured. In some embodiments, genetically engineered immune cells are administered to a subject followed by the administration of a composition as disclosed herein, thereby targeting the genetically engineered immune cells to a desired target tissue, such as a cancer.

In several embodiments, bi-functional compositions are delivered to a tumor or a cancerous tissue as a tumor-killing tool. In some embodiments, provided herein is a method of treating or otherwise managing a cancer or tumor, comprising: capturing anti-tumor cells with bi-functional compositions as disclosed herein, contacting the captured cells with the cancer or tumor by way of the bi-functional composition; and applying a magnetic field around or adjacent to the cancer or tumor. In some embodiments, the anti-tumor cell is a T cell, such as a CD8⁺ or CD4⁺ T cell, or a natural killer (NK) cell. Other embodiments provided herein can be used in combination with embolization, chemoembolization and/or chemotherapy.

Non-limiting examples of tumors or cancers which can be treated in accordance with the compositions and methods provided herein include, e.g., tumors or cancers of the kidney, lung, prostate, pancreas, stomach, colon, liver, brain, testes or ovaries, oropharynx, and bladder and can be benign or malignant. Representative examples of tumors or cancers include hepatocellular adenoma, cavernous haemangioma, focal nodular hyperplasia, bile duct adenomas, bile duct cystadenomas, fibromas, lipomas, leiomyomas, mesotheliomas, teratomas, myxomas, and nodular regenerative hyperplasia, hepatocellularcarcinoma, cholangiocarcinoma, angiosarcoma, cystadenocarcinoma, squamous cell carcinoma, hepatoblastoma, melanoma, Hodgkin's and non-Hodgkin's lymphoma, tumors of the breast, ovary, and prostate.

Other non-limiting examples of tumors or cancer that can be treated by the compositions and methods provided herein include acute lymphoblastic leukemia, acute myeloid leukemia, Ewing's sarcoma, gestational trophoblastic carcinoma, Hodgkin's disease, Burkitt's lymphoma diffuse large cell lymphoma, follicular mixed lymphoma, lymphoblastic lymphoma, rhabdomyo sarcoma, testicular carcinoma, wilms's tumor, anal carcinoma, bladder carcinoma breast carcinoma, chronic lymphocytic leukemia, chronic myelogenous leukemia, hairy cell leukemia, head and neck carcinoma, lung (small cell) carcinoma, multiple myeloma, follicular lymphoma, ovarian carcinoma, brain tumors (astrocytoma), cervical carcinoma, colorectal carcinoma, hepatocellular carcinoma, Kaposi's sarcoma, lung (non-small-cell) carcinoma, melanoma, pancreatic carcinoma, prostate carcinoma, soft tissue sarcoma, breast carcinoma, colorectal carcinoma (stage III), osteogenic sarcoma, ovarian carcinoma (stage III), testicular carcinoma, or combinations thereof.

In some embodiments, immune cells (e.g., phagocytes) are captured by the bifunctional compositions as disclosed herein and directed to targeted infectious tissue to clear up the insulting bacteria, fungal or viruses, synergizing the endogenous homing signals.

In several embodiments, phagocytes are captured by a first population of antibodies which are then subsequently directed, either with or without the additional application of a magnetic field, to a target tissue which is under the influences of an infection (e.g., bacteria, fungal, viral, etc), inflammation, or combinations thereof. Such phagocytes include, but are not limited to neutrophils, monocytes, macrophages, dendritic cells, mast cells (and in some embodiments, one or more cell types that perform phagocytosis as a non-primary function, including but not limited to epithelial cells, endothelial cells, fibroblasts, and mesenchymal cells). Thus, in several embodiments, the bifunctional compositions as disclosed herein provide synergistic results as compared to the body's endogenous homing signals alone.

As discussed above, in several embodiments, neural progenitor cells, are captured and delivered in several embodiments to treat neurodegenerative disorders. In some embodiments, however, progenitor cells are not captured and delivered, but rather neurotrophic cells are captured by antibodies coupled to magnetic particles and delivered to a neurodegenerative site. Such cells include, but in several embodiments, astrocytes and/or Schwann cells as well as other cells that support neural tissue. In some embodiments, exogenous neural cells (e.g., cultured neurons) are targeted molecularly, magnetically, or both, to the neurodegenerative tissue.

### Magnetic Particles

Magnetic particles as used in the compositions and methods provided herein can be in any forms known in the art, e.g., fluid (e.g., ferrofluid), microspheres, conjugates (e.g., poly-L-Lysine (“PLL”) conjugates), micelles, colloids, liposomes, aggregates, or complexes with a range of various sizes. In some embodiments, the diameter of a magnetic particle as used herein ranges from about 10 nm to about 20000 nm, from about 500 nm to about 7000 nm, from about 1000 nm to about 6000 nm, or from about 3000 nm to about 5000 nm, from about 300 nm to about 900 nm, or from about 50 nm to about 500 nm, and overlapping ranges thereof. In several embodiments, the diameter of a magnetic particle as used herein is about 900 nm. In other embodiments, the diameter ranges from about 10 nm to about 20 nm, about 15 nm to about 25 nm, about 20 nm to about 30 nm, about 25 nm to about 35 nm, about 30 nm to about 40 nm, about 35 nm to about, 45 nm, and overlapping ranges thereof. Any material that is responsive to a magnetic field can be used, for example, a ferromagnetic, paramagnetic or superparamagnetic material. In some embodiments, the magnetic particles are nanoparticles, e.g., superparamagnetic iron oxide (SPIO). In some embodiments, the magnetic particles are biodegradable, e.g., biodegradable magnetic microspheres. Magnetic particles as used herein can be obtained, for example, by spray drying magnetic material under gravity, or under the presence of an applied electric or magnetic field. Magnetic particles useful in the methods provided herein are also commercially available (e.g., Endorem, or Amag Pharmaceutical Inc.'s FERIDEX®, FERIDEX IV®), MACS® (MicroBeads; Miltenyi Biotec Inc., Auburn, Calif.), and other similar particles known in the art.

In several embodiments, magnetic particles that are approved by the FDA are used, such as, for example, FERAHEME® particles (also known as ferumoxytol (AMI-228)), were recently (Jun. 30, 2009) FDA-approved for treatment of iron deficiency anemia. FERAHEME® is a member of the class of monodispered nano-sized ultra-small superparamagnetic iron oxides (USPIO). It has a molecular weight of 731 kD and a hydrodynamic diameter of 30 nm. FERAHEME® is approved for the treatment of iron deficiency anemia in adult patients with chronic kidney disease. Advantageously, as discussed below, the carboxyl (COOH) groups on FERAHEME® particles enable covalent coupling of antibodies by activating the carboxyl groups with water-soluble carbodiimide (e.g. EDAC reagent). In some embodiments, the initially used particles are non-magnetic, weakly magnetic, or partially magnetic particles that are coupled with naturally occurring magnetically responsive proteins, for instance, ferritin conjugates.

### Antibodies and Markers

A variety of antibodies can be used in the compositions and methods disclosed herein. Depending on the therapeutic cell type and the target cell type antibodies can be directed against very unique markers, or against more generally expressed markers (e.g., those that identify a particular genus of stem cells, but not a specific species, such as, for example, mesenchymal stem cell markers, as opposed to adipose specific markers). In some embodiments, the markers against which the antibodies that capture therapeutic cells are directed include, but are not limited to c-kit, CD2, CD2a, CD 105, CD90, CD31, CD45, CD11a, CD54, CD68, flk-1, smooth muscle cell myosin heavy chain, vascular endothelial cadherin, bone specific alkaline phosphatase, hydroxyapatite, ostocalcin, bone morphogenic protein, CD4, CD8, CD34, CD38, CD44, colony forming unit (CFU), fibroblast colony-forming unit (CFU-F), CD45, Mac-1, Sca-1, Stro-1 antigen, Thy-1, adipocyte lipid-binding protein (ALBP), fatty acid transporter (FAT), albumin, beta-1 integrin, CD133, glial fibrillary acidic protein (GFAP), microtubule-associated protein-2 (MAP-2), myelin basic protein (MPB), nestin, neural tubulin, neurofilament (NF), noggin, O4, O1, synaptophysin, tau, alkaline phosphatase, embryonal carcinoma, alpha-fetoprotein (AFP), bone morphogenetic protein-4, brachyury, CD30, cripto (TDGF-1), GATA-4, GCTM-2, genesis, germ cell nuclear factor, hepatocyte nuclear factor-4 (HNF-4), neuronal cell-adhesion molecule (N-CAM), OCT4/POU5F1, Pax6, stage-specific embryonic antigen-3 (SSEA-3), stage-specific embryonic antigen-4 (SSEA-4), telomerase, TRA-1-60, TRA-1-81, vimentin, myoD, Pax7, myogenin, MR4, Myosin heavy chain, and myosin light chain. In several embodiments, one or more of these markers could be used as a marker to target a specific tissue (e.g., a damaged, injured or diseased tissue).

In several embodiments, magnetic particles are coupled to one population of antibodies that recognize cells that target cancers (as discussed above) and another population of antibodies that are directed to cancer cell-specific markers. For example, in one embodiment, a population of anti-CD54 antibodies capture CD54-positive cells and a second population of antibodies is directed to antigens expressed by prostate cancer cells, such as prostatic acid phosphatase (PAP). Presently, some anti-cancer approaches employ autologous cells which could target prostate cancer cells. In several embodiments, however, the combination of capturing specific therapeutic cells (e.g., B cells and killer T cells) and directing them to the target cancer cells (based on molecular targeting), including with enhancing delivery by employing a magnetic field provides unexpected increases in therapeutic efficacy. In other embodiments, other cancer-specific markers are used to direct cells to various types of cancer cells, including but not limited to ALK gene rearrangements to target non-small cell lung cancer and/or anaplastic large cell lymphoma; alpha-fetoprotein (AFP) to target liver cancer and/or germ cell tumors; beta-2-microglobulin (B2M) to target multiple myeloma, chronic lymphocytic leukemia, and certain lymphomas; beta-human chorionic gonadotropin (Beta-hCG) to target choriocarcinoma and/or testicular cancer; BCR-ABL to target chronic myeloid leukemia; BRAF mutations (e.g., V600E) to target cutaneous melanoma and/or colorectal cancer; CA15-3/CA27.29 to target breast cancer; CA19-9 to target pancreatic cancer, gallbladder cancer, bile duct cancer, and/or gastric cancer; CA-125 to target ovarian cancer; calcitonin to target medullary thyroid cancer; carcinoembryonic antigen (CEA) to target colorectal cancer and/or breast cancer; CD20 to target non-Hodgkin lymphoma; chromogranin A (CgA) to target neuroendocrine tumors; cytokeratin fragments 21-1 to monitor recurrence of lung cancer; EGFR mutants to target non-small cell lung cancer; Estrogen receptor (ER)/progesterone receptor (PR) to target breast cancer; fibrin/fibrinogen to target bladder cancer; HE4 to target ovarian cancer; HER2/neu to target breast cancer; gastric cancer; esophageal cancer; various immunoglobulins to target multiple myelomas and/or Waldenstrom macroglobulinemia; KIT to target gastrointestinal stromal tumor and/or mucosal melanoma; KRAS mutants to target colorectal cancer and/or non-small cell lung cancer; lactate dehydrogenase to target germ cell tumors; nuclear matrix protein 22 to target bladder cancer; thyroglobulin to target thyroid cancer; urokinase plasminogen activator (uPA) and/or plasminogen activator inhibitor (PAI-1) to target breast cancer; BRCA-1 and/or BRCA-2 to target breast cancer, TA-90, S-100. In several embodiments, certain of the markers listed above (or disclosed elsewhere herein) may also be expressed by normal (e.g., non-cancerous cells), however, in several embodiments, the expression is upregulated (or otherwise altered in a unique fashion) during cancer. As such, certain embodiments disclosed herein can, by supplementing the molecular targeting with magnetic targeting, further exploit the altered expression of one or more markers by cancerous cells to specifically target and neutralize (e.g., kill or reduce replication of) those cancerous cells.

Similarly, treatment of bacterial or viral infections may be enhanced by the embodiments disclosed herein. In some embodiments, antibodies against a particular infectious (e.g., a particular bacteria or virus) agent can be coupled to magnetic particles, allowing the capture of immune cells that target that agent, in conjunction with antibodies directed against a marker expressed by the infectious agent itself. By way of example, an antibody directed against activated lymphocytes can be captured by a first population of antibodies coupled to a magnetic particle, which in turn is coupled to an antibody directed inflammatory molecules (including but not limited to, for example, IL-6 (Interleukin-6), IL-8 (Interleukin-8), IL-18 (Interleukin-18), TNF-α (Tumor necrosis factor-alpha), CRP (C-reactive protein)). As a result, the molecular targeting will chaperone the activated lymphocyte to a site of infection/inflammation. In several embodiments, this effect is synergistically enhanced by the application of a magnetic field to further target and/or retain the therapeutic cells at the site of infection. Thus, rather than relying exclusively on endogenous homing signals, the innate signals are enhanced by the application of a magnetic field, supplementing the molecular targeting and leading to increased therapeutic effects.

In several embodiments, bacterial infections, including but not limited to those caused by one or more of the following genus of bacteria are targeted: Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, and Yersinia. In several embodiments, bacterial antigens that are recognized include, but are not limited to, lipoteichoic acid (LTA) for gram positive bacteria, Mycoplasma pneumonia, and/or chlamydia pneumoniae.

Similar approaches, in several embodiments are used in treating viral infections, including infections caused by one or more of adenovirus, Coxsackievirus, Epstein-Barr virus, hepatitis a virus, hepatitis b virus, hepatitis c virus, herpes simplex virus, type 1, herpes simplex virus, type 2, cytomegalovirus, ebola virus, human herpesvirus, type 8, HIV, influenza virus, measles virus, mumps virus, human papillomavirus, parainfluenza virus, poliovirus, rabies virus, respiratory syncytial virus, rubella virus, and varicella-zoster virus.

Antibodies against the markers listed above (or disclosed elsewhere herein) can be polyclonal or monoclonal, and raised in any appropriate organism that generates a sufficient interaction with marker, without adverse (or with minimized) immune responses from the recipient. Depending on the embodiment, the antibodies may be of the IgA, IgD, IgE, IgG or IgM istotype. In some embodiments, combinations of antibodies may be used to capture even more specific subpopulations of therapeutic cells (e.g., c-kit positive and GATA-4 positive). In some embodiments, antibodies on the magnetic particles that recognize integrin, fibronectin, and/or tissue factor(s) allow targeting of more cell types and allow selection of cell populations with lower specificity.

With respect to markers on the target tissues, again, unique or context specific markers may be used, depending on the embodiment. For example, in the context of cardiac repair, a general cardiac antibody could be employed, in one embodiment, in order to generally direct the composition to the heart. In certain such embodiments, the subsequent application of a magnetic field can be used to more specifically localize the targeting of the composition. In some embodiments, a cardiac specific maker that is upregulated in response to injury may be chosen. For example, proinflammatory cytokines such as IL-1beta and IL-6 are upregulated shortly after myocardial infarction. Other inflammatory markers, such as, for example, tumor necrosis factor, IL-10, MCP-1, MIP-1 are targeted with the second population of antibodies. In several embodiments, pro-angiogenic genes, such as IL-8, FGF-2, VEGF-R2 are targeted with the second population of antibodies. Cytokines are also used as markers, in several embodiments, such as for example, SDF-1, G-CSF, GM-CSF, CXCR-4, transforming growth factor, IGF or HGF are targeted with the second population of antibodies. In some embodiments, cells expressing myosin light chain are targeted. As such, the second antibody can be used to selectively target damaged myocardial tissue. In still additional embodiments, apoptotic surface markers, such as Fas (e.g. CD 95) could be targeted with the antibody. Depending on the embodiment, any of the target tissue markers (whether cardiac or otherwise) can be used in combination with any of the antibodies directed against markers on therapeutic cells. Also, depending on the embodiment, a single population of antibodies can recognize a marker that serves to facilitate the delivery of therapeutic cells to a target site (e.g., the therapeutic cells and the target tissue share a marker) in conjunction with a magnetic field (or in some embodiments, without a magnetic field).

Other specific markers for other organs are used in other embodiments. Likewise, many damage or disease-specific markers known in the art can be used to specifically target the compositions to a particular organ, or in some embodiments, to a particular region of an organ. As discussed, above, certain cancer-specific, virus-specific, bacterial specific, or inflammation specific markers (or other identifying characteristics of target cells) are targeted by a population of antibodies coupled to magnetic particles, thereby allowing the enhanced delivery (supplementation of molecular targeting with magnetic targeting) of a population of therapeutic cells coupled to another population of antibodies coupled to the magnetic particles.

Additionally, exogenous markers may be exploited, either on the therapeutic cell side or the target tissue side. For example, a particular unique marker could be introduced into a subject that becomes integrated into all of the subject's stem cells expressing markers of potential cardiac differentiation. As a result, this particular marker could be targeted by one of the antibody populations on the magnetic particle, thereby increasing the percentage of therapeutic cells that are known to have the capacity to generate cardiac tissue.

In some embodiments, single bi-functional antibodies linked to a magnetic particle can be used in place of two distinct antibodies. The recognition components of the bi-functional antibodies may be selected from any one of a naturally occurring, synthetic or recombinant antibody, single chain Fv (scFv), bi-functional scFv, diabody, F(ab) unit, F(ab′) unit, bi-specific F(ab′) conjugate, chemically cross-linked bi-functional antibody, linear antibody, F(ab′)2 antigen binding fragment of an antibody, or any functional fragments thereof. Preferably the recognition component is a bi-functional F(ab′) conjugate, e.g., two F(ab′) units linked together. In still additional embodiments, drugs or other pharmaceuticals may be used in place of therapeutic cells. In still further embodiments, the magnetic particles are also used for visualization of the therapeutic cells or agents, while in other embodiments, the magnetic particles are not visualized.

### Methods for Coupling Antibodies and Magnetic Particles

In some embodiments, certain magnetic particles have various properties that enhance the ability to couple the various antibody populations to the particles. For example, FERAHEME® has a carboxylated polymer coating. The carboxyl (COOH) groups on FERAHEME® particles allow covalent coupling of antibodies by activating the carboxyl groups with water-soluble carbodiimide (e.g., EDAC reagent). The EDAC reacts with the carboxyl group to create an active ester that is reactive toward primary amines on certain types of antibodies. As such, through a chemical reaction, the antibodies for the therapeutic and target cells can be coupled to the magnetic particle.

In some embodiments, various polymer coatings can be used to tailor the way in which the antibodies can be coupled to the particles. For example, the particles may be coated with a biotinylated polymer which could be reacted with antibodies that have had a streptavidin moiety incorporated. Such embodiments offer an additional advantage in that the particles (or the antibodies) could be prepared with an excess of biotin (or streptavidin), that could allow an additional modality for assessing the targeting of the composition to the target tissue.

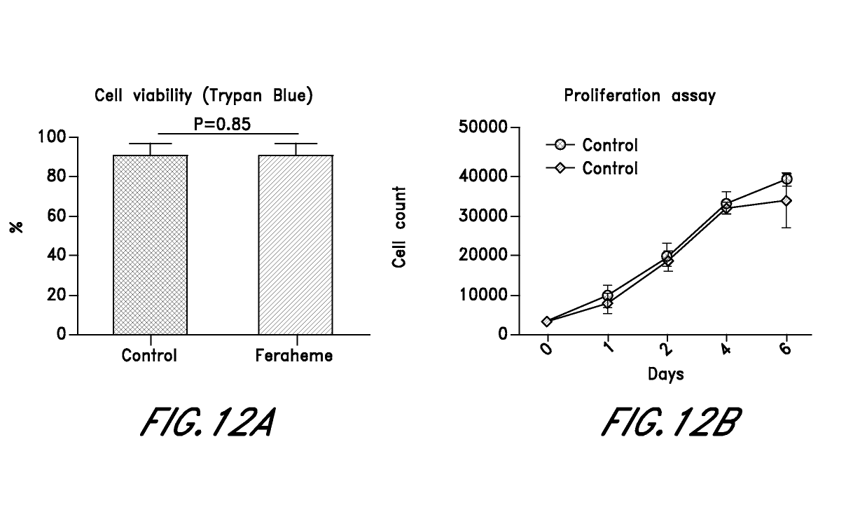

Prior experiments have demonstrated that the labeling of cells themselves, which is used in several embodiments, with magnetic particles has limited adverse effects on the cells (see, e.g., FIGS. 12A-12B). In some instances, cell viability is substantially maintained during and after the labeling protocol. In some embodiments, labeling does not affect the antigenic phenotype or proliferation of the labeled cells. In some embodiments, modest increases in apoptosis of cells occur during the labeling process, however, in some such embodiments, necrosis of the cells is lessened. As such, a healthy and viable labeled cell population is produced according to several of the labeling embodiments described herein. As a result, it is believed that the capture of cells via antibodies linked to a magnetic particle will have no (or limited) adverse effects on the cells, thereby enabling the cells to contribute to the repair and/or regeneration of the target tissue.

### Target Tissues

The target tissues can be any tissue of a subject that is in need of inhibition and/or elimination (e.g., in the case of cancers or infections) repair, regeneration and/or diagnosis. For example, cancerous tissue could be targeted with compositions disclosed herein, in order to accumulate a population of cancer-killing cells (e.g. cytotoxic T cells) to the local area of tumor tissue. As another example, the cardiac tissue of a subject recently having an adverse cardiac event could be targeted with the compositions disclosed herein, in order to deliver a population of therapeutic stem cells directly to the local area of the cardiac tissue that had been damaged. In several embodiments, the brain and other neural tissues, lungs, blood vessels, liver, kidneys, intestines, spleen, pancreas, could all be selected as target tissues (or sources of therapeutic cells).

For example, in some embodiments, the tissue or organ is or is part of the lymphatic system, liver, spleen, pancreas, heart, urogenital tract, gastrointestinal tract, respiratory system, portal venous system, ventricular fluid system, or cerebrospinal fluid system. In some embodiments, the tissue or organ for which delivery or treatment is desired comprises cancerous areas, areas of atherosclerosis, areas of post-angioplasty restenosis, areas of plaque fracture, sites of thrombosis or sites of vasculitis. In some embodiments, the tissue or organ comprises gravity-dependent and gravity-independent areas. In some embodiments, the target tissue or organ comprises a luminal surface.

In one embodiment, cardiac cells, endothelial cells, fibroblasts or smooth muscle cells are captured by the bi-functional composition and directed to the heart, e.g., to diseased or injured cardiac tissue. In some embodiments, endothelial cells, fibroblasts or hepatocytes are captured by the bi-functional composition and directed to the liver to treat hepatic disease. In one embodiment, magnetic particle-labeled neural cells, neuroglial cells, endothelial cells or fibroblasts are captured by the bi-functional composition and directed to the brain or spinal cord. In one embodiment, endothelial cells, fibroblasts, pancreatic islet cells or other pancreatic cells are captured by the bi-functional composition and directed to the pancreas. In some embodiments, endothelial cells, fibroblasts or respiratory epithelial cells are captured by the bi-functional composition and directed to the lung or respiratory system. In one embodiment, endothelial cells, smooth muscle cells or fibroblasts are captured by the bi-functional composition and directed to blood vessels, e.g., atherosclerotic vessels. In another embodiment, endothelial cells, epithelial cells, fibroblasts or smooth muscle cells are captured by the bi-functional composition and directed to gastrointestinal or urogenital tissues.

As discussed above, tissue-specific or injury-specific markers are selected, in some embodiments to further enhance the magnetic targeting of the compositions to the desired tissue location. Targeting of the compositions in two respects improves, synergistically in several embodiments, the therapeutic efficacy of the compositions. Magnetic targeting in several embodiments, serves to grossly localize cells to a particular tissue. For example, the tailored positioning of a magnetic field (or fields) allows for targeting to the liver, the stomach, the lungs, or the heart, as non-limiting examples. Thus, magnetic targeting allows the localization of the therapeutic compositions to an organ of interest, and reduces and/or minimizes the delivery of the therapeutic compositions to a non-target organ. In several embodiments, the magnetic targeting enables localization of the therapeutic compositions to a particular subregion of a target organ (e.g., the left ventricle of the heart, a particular lobe of the liver, etc.). In conjunction with the magnetic targeting, antibody targeting enables a fine-tuning of the ultimate target for the therapeutic composition (e.g., further localization within the target tissue can occur). In several embodiments, the antibodies are selected such that the target antigen is exposed, expressed, or otherwise available to interact with the antibody only during or after an injury, disease or other adverse event. Thus, in combination, magnetic targeting and antibody targeting result in highly precise targeting to specific regions of tissues that are in need of therapeutic repair. Not only does this combination, in several embodiments, result in synergistically improved therapeutic effects, but it also reduces possible adverse effects that could arise if the therapeutic composition were to be delivered and act at a non-target (e.g., non-injured and/or non-diseased site).

### Magnetic Fields

In some embodiments, the bi-functional magnetic compositions are guided towards the target tissue or organ by one or more magnetic fields or magnetic field gradients (e.g., an external source of magnetic fields or magnetic field gradients). Such fields or gradients can be generated by, for example, one or more magnets and/or associated medical devices placed within or adjacent to a target tissue or organ prior to, during or after delivery of the composition. In some embodiments, the magnets are placed inside the body using surgical or percutaneous methods, inside the target tissue, or outside the target tissue (e.g., around or adjacent to the target tissue). In some embodiments, the magnets are external magnets that are placed outside of a subject's body to create an external source of magnetic field around or adjacent to the target tissue or organ. In some embodiments, the source of magnetic fields is a permanent magnet (e.g., neodymium (NdFeB) magnet). In one embodiment, the source of magnetic fields is an electro-magnet. In other embodiments, the size of magnets ranges from about 1 mm to about 10 m and the strength of magnetic fields ranges from about 0.1 Tesla to about 100 Tesla, including about 0.1 to about 0.5 Tesla, about 0.5 to about 1 Tesla, about 1 Tesla to about 1.1 Tesla, about 1.1 Tesla to about 1.2 Tesla, about 1.2 Tesla to about 1.3 Tesla, about 1.3 Tesla to about 1.4 Tesla, about 1.4 Tesla to about 1.5 Tesla, about 1.5 Tesla to about 2 Tesla, about 2 Tesla to about 4 Tesla, about 4 Tesla to about 10 Tesla, about 10 Tesla to about 30 Tesla, about 30 Tesla to about 50 Tesla, about 50 Tesla to about 70 Tesla, about 70 Tesla to about 90 Tesla, and overlapping ranges thereof. In several embodiments, the magnetic field is applied for a time period ranging from about 1 minute up to about 5 hours. In some embodiments, the magnetic field is applied for about 1 minute to 5 minutes, about 5 minutes to about 10 minutes, about 10 minutes to about 20, about 20 minutes to about 30 minutes, and overlapping ranges thereof. In several embodiments, the magnetic field is applied for about 5-15 minutes, including about 6, 7, 8, 9, 10, 11, 12, 13, or 14 minutes.

In some embodiments, the source of magnetic fields is from one or more magnets of an apparatus (e.g., a group of magnets as an integral apparatus to shape and focus the magnetic field). Such apparatus can include, for example, a surgical tool (e.g., catheters, guidewires, and secondary tools such as lasers and balloons, biopsy needles, endoscopy probes, and similar devices) with a magnetic tip attachment. Thus in some embodiments, the bifunctional composition is delivered and an external magnet is used to target the composition (e.g., percutaneous or surgical access to the heart for cell injection with a magnet placed on the chest of a human subject). In some embodiments, the composition is delivered endomyocardially and a magnet is used to retain the composition within target site of the heart for a period of time sufficient to allow interaction of the target tissue antibodies and the therapeutic cell antibodies to interact with their respective antigens.

In several embodiments, the delivery and targeting means are combined. For example, in some embodiments, specialized catheters are used to deliver the composition and generate a localized magnetic field that functions to enhance retention of the composition at a desired target area. In some embodiments, the catheter also comprises a screw-like tip (or other shape) that allows for reversible anchoring of the catheter in tissue at the target site. Other reversible anchors may be used, such as pinchers, retractable barbs and the like. In some embodiments, the anchored tip is advantageous because the delivery can occur while the heart is beating, and the anchored tip assists in stabilizing the tip against the moving cardiac tissue. In several embodiments, the catheter is also steerable, in order to allow navigation from a remote site to the desired region of a target tissue (e.g., from a femoral access point to the endomyocardial wall). In some embodiments, the catheter comprises a controller that allows an operator to initiate the generation of a magnetic field. In some embodiments, a specific strength of magnetic field can be generated. In some embodiments, the magnetizable portion is distinct from the delivery tip, while in other embodiments, the delivery tip is housed in or adjacent to, the magnetizable portion. In some embodiments, the catheter comprised a delivery lumen that is of sufficient size to allow free passage of the composition from the lumen to the target site. For example, the diameter of the delivery lumen, in some embodiments, ranges from about 25 to about 50 microns, about 50 to about 100 microns, about 100 microns, to about 200 microns, about 200 to about 300 microns, about 300 to about 400 microns, and overlapping ranges thereof. In some embodiments, the presence of the magnetic field enhances the efflux of the composition from the catheter (e.g., minimizes residual, undelivered particles).

In some embodiments, the magnetic fields function primarily to target the cells to a desired location. However, in some embodiments, the magnetic fields play ancillary roles. For example, in some embodiments, the magnetic field, in conjunction with the magnetic particles, is used for imaging or visualization of the particles. In other embodiments, however, visualization or tracking is not performed, or is performed at a later time. As another example, in some embodiments, magnetic fields also inhibit the induction and or progression of apoptosis, which further increases the efficacy of the therapeutic cells.

In some embodiments, a magnetic resonance imaging (MRI) instrument or equivalent may be used to shape or focus the magnetic field, with or without the use of a local magnetic coil within the MRI field to enhance targeting. In some embodiments, computer simulations can aid magnet designs for acquiring optimal magnetic field strength to capture the bi-functional compositions. For example, fluid flow rate, cell/magnetic particle size, antibody-antigen interaction strength, iron oxide content, distance of magnet from the target tissue, and/or other variables are considered in some embodiments when determining the strength and need for focusing of the magnetic field. In some embodiments, the magnet designs can be assessed in an in vitro flow system by placing labeled cells in peristaltic pump-driven flow. In some embodiments, focusing of the magnetic field is not performed.

In several embodiments, magnetic targeting is reduced and in some cases eliminated, thus using antibody targeting alone (without magnetic targeting). Such embodiments are used when a subject has one or more contraindications to exposure to a magnetic field. For example, in several embodiments, a patient with a heart pacemaker may face serious adverse consequences if exposed to a magnetic field (e.g., malfunction of the pacemaker). Patients who have implants (e.g., insulin pumps, neurostimulators, cochlear implants, and the like) that would be exposed to the magnetic field during delivery of the therapeutic composition, may suffer adverse consequences. Danger of de-programming or dislodging of such devices is possible. However, in several embodiments, focusing of the magnetic field to a specific location and/or adjusting the parameters of the magnetic field may reduce or eliminate concerns about such contraindications.

In some embodiments, tools such as injection needles, balloons, catheters or other acceptable delivery devices are used to deliver labeled cells. In some embodiments, the targeting magnet is placed at desired locations via a fiberoptic tube or catheter. In some embodiments, the catheter or interventional device tip is guided or monitored by a control system (e.g., a radar-assisted system or a real-time localization system) to provide more precise localization of the administered cells (see, e.g., U.S. Pat. No. 7,280,863; herein incorporated by reference). In some embodiments, a catheter Guidance Control and Imaging (GCI) apparatus is used to position and fixate a catheter, and to view the catheters' position with the x-ray imagery overlaying the display (see e.g., PCT Publ. Nos. WO 2004/006795 and 2005/042053; herein incorporated by reference). In one embodiment, such an apparatus can include, for example, an operator control that possesses a positional relationship to the catheter tip in addition to being a model representation of the actual or physical catheter tip advancing within the patient's body. In another embodiment, the physical catheter tip (the distal end of the catheter) of such apparatus can include a permanent magnet that responds to a magnetic field generated externally to the patients body (see e.g., U.S. Pat. Publ. Nos. 2004/0019447, 2006/0114088, 2006/0116633 and 2006/0116634; herein incorporated by reference). In yet another embodiment, such apparatus can include magnetic sensors to detect the magnetic field of generated by the catheter tip. In some embodiments, each sensor transmits the field magnitude and direction to a detection unit, which filters the signals and removes other field sources. The method allows the measurements of magnitude corresponding to the catheter tip distance from the sensor and the orientation of the field showing the magnetic tip orientation (see e.g., U.S. Pat. Publ. 2008/0249395; herein incorporated by reference).

In several embodiments, the magnetic field(s) are generated transiently during and after the delivery of the bi-functional compositions. For example, in some embodiments, a magnetic field is generated just prior to the inception of delivery and maintained for several minutes after delivery. In several embodiments, the magnetic field is maintained for about 2 to about 5 minutes, about 3 to about 6 minutes, about 4 to about 7 minutes, about 5 to about 8 minutes, about 6 to about 9 minutes, or about 7 to about 10 minutes. In some embodiments, the field(s) is maintained for about 5 minutes to about 10 minutes, about 10 minutes to about 15 minutes, about 15 minutes to about 20 minutes, about 20 minutes to about 25 minutes, and overlapping ranges thereof. Longer exposure to the magnetic field is used in embodiments wherein a larger dose of the bi-functional composition has been delivered and/or wherein the region of damaged tissue is particularly large.

In some embodiments, an implant is employed to facilitate delivery and retention of the bi-functional compositions in the target tissue or organ. In such embodiments, the bi-functional composition is delivered from a catheter or an interventional device distal tip to a previously placed implant (e.g., stents). In some embodiments, the implants are labeled by application of a magnetic field sequence. In these embodiments, labeled cells can be attracted by the local magnetic domains and associated field gradients within the implant, and attach onto the tissue structures locally protruding through the stent or implant struts.

In some embodiments of the methods provided herein, the bi-functional compositions are delivered to (or otherwise contacted with) a cardiac tissue. For example, in some embodiments, the bi-functional compositions are delivered systemically (or locally) and targeted to the heart, including specific anatomical regions of the heart. In some embodiments, bi-functional compositions are delivered locally and targeted to a specific region of the heart (and capture the therapeutic cells passing by through the circulation). In some embodiments, the bi-functional compositions are directly injected epicardially into a cardiac tissue, for example, during an open chest surgery. In other embodiments, the bi-functional compositions are delivered to a cardiac tissue using non-surgical methods (e.g., minimally invasive interventions). Such methods include, for example, intravascular (e.g., intracoronary or intravenous) or intramyocardial administration. In some embodiments, the bi-functional compositions are delivered to a tissue via intracoronary infusion followed by administration of exogenous therapeutic cells (for example, CDCs, e.g., autologous CDCs). In some embodiments, administration of exogenous cells is not necessary, as endogenous cell capture/recruitment is sufficient for significant therapeutic benefits. In some embodiments, despite relatively high blood flow rates in the target tissue, the combination of magnetic and molecular targeting results in significant retention of therapeutic cells in the target tissue. In some embodiments, bi-functional compositions are prepared for administration by mixing, admixing, or compounding the compositions within an injectable liquid suspension or any other biocompatible medium.

In some embodiments, catheters are advanced through the vasculature and into the target tissue to deliver the bi-functional compositions. In several embodiments, intravenous administration routes are used, either by continuous drip or as a bolus. In yet another embodiment, wherein cardiac tissue is the target tissue, the bi-functional compositions are administered to the cardiac tissue by intramyocardial administration, for example, using a conventional intracardiac syringe or a controllable endoscopic delivery device having a needle lumen or bore of sufficient diameter to reduce shear forces that could damage the bi-functional compositions. In other embodiments, the bi-functional compositions are administered to the cardiac tissue using an endocardial approach that delivers materials into the cardiac wall from within the chamber of the heart (e.g., endomyocardial procedure).

In some embodiments of the methods provided herein, the bi-functional compositions are administered to the peri-infarct zone of cardiac tissue that was subject to an infarction. In some embodiments, the bi-functional compositions are administered in a system, e.g., long-term, short-term and/or controlled release system, which can improve cell engraftment and persistence. In some embodiments, the system is a matrix, such as a natural or synthetic matrix. The matrix can function to retain the bi-functional compositions in place at the site of injury by serving as scaffolding, which in turn enhances the opportunity for the interaction of the bi-functional composition with the target tissue and/or with the therapeutic cell population.

In some embodiments, the bi-functional compositions are administered to the subject once. In other embodiments, bi-functional compositions are administered to the subject more than one time (e.g., as in an ongoing therapeutic regimen). In several embodiments, a series of administrations occurs, with monitoring of the functionality of recipient's target organ being used to determine if and when an additional administration of bi-functional composition is needed. For example, in several embodiments, bi-functional compositions are administered 2-5 times, 5-10 times, 10-20 times, 20-30 times, or more.

An effective dose of labeled bi-functional composition will vary depending on the therapeutic cell type, the target tissue, the degree of antibody conjugation to the magnetic particles, the strength and/or focus of the magnetic field, and other clinically relevant variables. In some embodiments, a dose of between about 1 and about 20 mg iron/kg body weight is used, including about 1 to about 2 mg iron/kg, about 2 to about 3 mg iron/kg, about 4 to about 6 mg iron/kg, about 6 to about 8 mg iron/kg, about 8 to about 10 mg iron/kg, about 10 to about 12 mg iron/kg, about 12 to about 14 mg iron/kg, about 14 to about 16 mg iron/kg, about 16 to about 18 mg iron/kg, about 18 to about 20 mg iron/kg, and overlapping ranges thereof. In other embodiments, dose is determined based on the capacity of the bi-functional composition to capture therapeutic cells. In some embodiments, the “dose” is sufficient to achieve the delivery to a target tissue of the equivalent of a dose of between about 1×10⁴ and about 1×10¹⁰ therapeutic cells, including about 1×10⁴ to about 1×10⁵, about 1×10⁵ to about 1×10⁶, about 1×10⁶ to about 1×10⁷, about 1×10⁷ to about 1×10⁸, about 1×10⁸ to about 1×10⁹, about 1×10⁹ to about 1×10¹⁰, and overlapping ranges thereof. Patient age, general condition, and immunological status may also be used as factors in determining the dose administered, and will be readily determined by the physician.

As discussed above, in some embodiments, one or more additional therapeutic agents, either alone or in combination with the bi-functional composition can be delivered systemically or locally to the target tissue. Non-limiting examples of therapeutic agents that can be used in combination with the bi-functional compositions, methods or kits provided herein include one or more of anti-neoplastic drugs, anti-angiogenesis drugs, pro-angiogenesis drugs, anti-fungal drugs, anti-viral drugs, anti-inflammatory drugs, anti-bacterial drugs, cytotoxic drugs, a chemotherapeutic or pain relieving drug and/or an anti-histamine drug. The agent can also be, for example, anyone or more of hormones, steroids, vitamins, cytokines, chemokines, growth factors, interleukins, enzymes, anti-allergenic agents, circulatory drugs, anti-tubercular agents, anti-anginal agents, anti-protozoan agents, anti-rheumatic agents, narcotics, cardiac glycoside agents, sedatives, local anesthetic agents, general anesthetic agents, and combinations thereof. In some embodiments, the therapeutic agent is an anti-neoplastic, chemotherapeutic or pain relieving drug.

Examples of anti-angiogenic or anti-neoplastic drugs include, without limitation, alkylating agents, nitrogen mustards, antimetabolites, gonadotropin releasing hormone antagonists, androgens, antiandrogens, antiestrogens, estrogens, and combinations thereof. Examples include but are not limited to actinomycin D, aldesleukin, alemtuzumab, alitretinoin, allopurinol, altretamine, amifostine, aminoglutehimide, amphotercin B, amsacrine, anastrozole, ansamitocin, arabinosyl adenine, arsenic trioxide, asparaginase, aspariginase Erwinia, BCG Live, benzamide, bevacizumab, bexarotene, bleomycin, 3-bromopyruvate, busulfan, calusterone, capecitabine, carboplatin, carzelesin, carmustine, celecoxib, chlorambucil, cisplatin, cladribine, cyclophosphamide, cytarabine, cytosine arabinoside, dacarbazine, dactinomycin, darbepoetin alfa, daunorubicin, daunomycin, denileukin diftitox, dexrazoxane, dexamethosone, docetaxel, doxorubicin, dromostanolone, epirubicin, epoetin alfa, estramustine, estramustine, etoposide, VP-16, exemestane, filgrastim, floxuridine, fludarabine, fluorouracil (5-FU), flutamide, fulvestrant, demcitabine, gemcitabine, gemtuzumab, goserelin acetate, hydroxyurea, ibritumomab, idarubicin, ifosfamide, imatinib, interferon (e.g., interferon α-2a, interferon α-2b), irinotecan, letrozole, leucovorin, leuprolide, lomustine, meciorthamine, megestrol, melphalan (e.g., PAM, L-PAM or phenylalanine mustard), mercaptopurine, mercaptopolylysine, mesna, mesylate, methotrexate, methoxsalen, mithramycin, mitomycin, mitotane, mitoxantrone, nandrolone phenpropionate, nolvadex, oprelvekin, oxaliplatin, paclitaxel, pamidronate sodium, pegademase, pegaspargase, pegfilgrastim, pentostatin, pipobroman, plicamycin, porfimer sodium, procarbazine, quinacrine, raltitrexed, rasburicase, riboside, rituximab, sargramostim, spiroplatin, streptozocin, tamoxifen, tegafur-uracil, temozolomide, teniposide, testolactone, tioguanine, thiotepa, tissue plasminogen activator, topotecan, toremifene, tositumomab, trastuzumab, treosulfan, tretinoin, trilostane valrubicin, vinblastine, vincristine, vindesine, vinorelbine, zoledronate, salts thereof, or mixtures thereof. In some embodiments, the platinum compound is spiroplatin, cisplatin, or carboplatin. In some embodiments, the drug is cisplatin, mitomycin, paclitaxel, tamoxifen, doxorubicin, tamoxifen, or mixtures thereof.

Other anti-angiogenic or anti-neoplastic drugs include, but are not limited to AGM-1470 (TNP-470), angiostatic steroids, angiostatin, antibodies against avβ3, antibodies against bFGF, antibodies against IL-I, antibodies against TNF-α, antibodies against VEGF, auranofin, azathioprine, BB-94 and BB-2516, basic FOF-soluble receptor, carboxyamido-trizole (CAI), cartilage-derived inhibitor (CDI), chitin, chloroquine, CM 101, cortisonelheparin, cortisonelhyaluroflan, cortexolonelheparin, CT-2584, cyclophosphamide, cyclosporin A, dexamethasone, diclofenaclhyaluronan, eosinophilic major basic protein, fibronectin peptides, Glioma-derived angiogenesis inhibitory factor (GD-AIF), GM 1474, gold chloride, gold thiomalate, heparinases, hyaluronan (high and low molecular-weight species), hydrocortisonelbeta-cyclodextran, ibuprofen, indomethacin, interferon-α, interferon γ-inducible protein 10, interferon-γ, IL-1, IL-2, IL-4, IL-12, laminin, levamisole, linomide, LM609, martmastat (BB-2516), medroxyprogesterone, methotrexate, minocycline, nitric oxide, octreotide (somatostatin analogue), D-penicillamine, pentosan polysulfate, placental proliferin-related protein, placental RNase inhibitor, plasminogen activator inhibitor (PAIs), platelet factor-4 (PF4), prednisolone, prolactin (16-kDa fragment), proliferin-related protein, prostaglandin synthase inhibitor, protamine, retinoids, somatostatin, substance P, suramin, SU101, tecogalan sodium (05-4152), tetrahydrocortisol-sthrombospondins (TSPs), tissue inhibitor of metalloproteinases (TIMP 1,2,3), thalidomide, 3-aminothalidomide, 3-hydroxythalidomide, metabolites or hydrolysis products of thalidomide, 3-aminothalidomide, 3-hydroxythalidomide, vitamin A and vitreous fluids. In another embodiment, the anti-angiogenic agent is selected from the group consisting of thalidomide, 3-aminothalidomide, 3-hydroxythalidomide and metabolites or hydrolysis products of thalidomide, 3-aminothalidomide, 3-hydroxy thalidomide. In one embodiment, the anti-angiogenic agent is thalidomide.

Examples of pain reliving drugs are, without limitation, analgesics or anti-inflammatories, such as non-steroidal anti-inflammatory drugs (NSAID), ibuprofen, ketoprofen, dexketoprofen, phenyltoloxamine, chlorpheniramine, furbiprofen, vioxx, celebrex, bexxstar, nabumetone, aspirin, codeine, codeine phosphate, acetaminophen, paracetamol, xylocalne, and naproxin. In some embodiments, the pain relieving drug is an opioid. Opioids are commonly prescribed because of their effective analgesic, or pain relieving, properties. Among the compounds that fall within this class include narcotics, such as morphine, codeine, and related medications. Other examples of opioids include oxycodone, propoxyphene, hydrocodone, hydromorphone, and meperidine. Narcotics, include, for example, without limitation, paregoric and opiates, such as codeine, heroin, methadone, morphine and opium.

Hormones and steroids, include, for example, without limitation, growth hormone, melanocyte stimulating hormone, adrenocortiotropic hormone, dexamethasone, dexamethasone acetate, dexamethasone sodium phosphate, cortisone, cortisone acetate, hydrocortisone, hydrocortisone acetate, hydrocortisone cypionate, hydrocortisone sodium phosphate, hydrocortisone sodium succinate, prednisone, prednisolone, prednisolone acetate, prednisolone sodium phosphate, prednisolone tebutate, prednisolone pivalate, triamcinolone, triamcinolone acetonide, triamcinolonehexacetonide, triamcinolone acetate, methylprednisolone, methylprednisolone acetate, methylprednisolone sodium succinate, flunsolide, beclomethasone dipropionate, betamethasone sodium phosphate, betamethasone, vetamethasone disodium phosphate, vetamethasone sodium phosphate, betamethasone acetate, betamethasone disodium phosphate, chloroprednisone acetate, corticosterone, desoxycorticosterone, desoxycorticosterone acetate, desoxycorticosterone pivalate, desoximethasone, estradiol, fludrocortisone, fludrocortisoneacetate, dichlorisone acetate, fluorohydrocortisone, fluorometholone, fluprednisolone, paramethasone, paramethasone acetate, androsterone, fluoxymesterone, aldosterone, methandrostenolone, methylandrostenediol, methyl testosterone, norethandrolone, testosterone, testosteroneenanthate, testosterone propionate, equilenin, equilin, estradiol benzoate, estradiol dipropionate, estriol, estrone, estrone benzoate, acetoxypregnenolone, anagestone acetate, chlormadinone acetate, flurogestone acetate, hydroxymethylprogesterone, hydroxymethylprogesterone acetate, hydroxyprogesterone, hydroxyprogesterone acetate, hydroxyprogesterone caproate, melengestrol acetate, normethisterone, pregnenolone, progesterone, ethynyl estradiol, mestranol, dimethisterone, ethisterone, ethynodiol diacetate, norethindrone, norethindrone acetate, norethisterone, fluocinolone acetonide, flurandrenolone, flunisolide, hydrocortisone sodium succinate, methylprednisolone sodium succinate, prednisolone phosphate sodium, triamcinolone acetonide, hydroxydione sodium spironolactone, oxandrolone, oxymetholone, prometholone, testosterone cypionate, testosterone phenyl acetate, estradiol cypionate, and norethynodrel.

Peptides and peptide analogs, include, for example, without limitation, manganese super oxide dismutase, tissue plasminogen activator (t-PA), glutathione, insulin, dopamine, peptide ligands containing RGD, AGD, RGE, KGD, KGE or KQAGDV (peptides with affinity for the GPEXma receptor), opiate peptides, enkephalins, endorphins and their analogs, human chorionic gonadotropin (HCG), corticotropin release factor (CRF), cholecystokinins and their analogs, bradykinins and their analogs and promoters and inhibitors, elastins, vasopressins, pepsins, glucagon, substance P, integrins, captopril, enalapril, lisinopril and other ACE inhibitors, adrenocorticotropic hormone (ACTH), oxytocin, calcitonins, IgG or fragments thereof, IgA or fragments thereof, IgM or fragments thereof, ligands for Effector Cell Protease Receptors (all subtypes), thrombin, streptokinase, urokinase, t-PA and all active fragments or analogs, Protein Kinase C and its binding ligands, interferons (α-IFN, β-IFN, γ-IFN), colony stimulating factors (CSF), granulocyte colony stimulating factors (GCSF), granulocyte macrophage colony stimulating factors (GM-CSF), tumor necrosis factors (TNF), nerve growth factors (NGF), platelet derived growth factors, lymphotoxin, epidermal growth factors, fibroblast growth factors, vascular endothelial cell growth factors, erythropoietin, transforming growth factors, oncostatin M, interleukins (IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, etc.), metalloprotein kinase ligands, collagenases and agonists and antagonists.

Antibodies, include, for example, without limitation, substantially purified antibodies or fragments thereof, including non-human antibodies or fragments thereof, and/or genetically engineered (e.g., recombinant) antibodies. In various embodiments, the substantially purified antibodies or fragments thereof, can be human, nonhuman, chimeric and/or humanized antibodies. Such non-human antibodies can be goat, mouse, sheep, horse, chicken, rabbit, or rat antibodies. The antibodies can be monoclonal or polyclonal antibodies.

Anti-mitotic factors include, without limitation, estramustine and its phosphorylated derivative, estramustine-phosphate, doxorubicin, amphethinile, combretastatin A4, and colchicine.

Anti-coagulation agents, include, for example, without limitation, phenprocoumon and heparin.

Circulatory drugs, include, for example, without limitation, propranolol.

Anti-viral agents, include, for example, without limitation, acyclovir, amantadine azidothymidine (AZT or Zidovudine), ribavirin, and vidarabine monohydrate (adenine arabino side, ara-A).

Anti-anginal agents, include, for example, without limitation, diltiazem, nifedipine, verapamil, erythritol tetranitrate, isosorbide dinitrate, nitroglycerin (glyceryl trinitrate), and pentaerythritolteiranitrate.

Antibiotics, include, for example, dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin G, penicillin V, ticarcillin, rifampin, and tetracycline.

Anti-inflammatory agents and analgesics, include, for example, diflunisal, ibuprofen, indomethacin, meclofenamate, mefenamic acid, naproxen, oxyphenbutazone, phenylbutazone, piroxicam, sulindac, tolmetin, aspirin and salicylates.

Cardiac glycoside agents, include, for example, without limitation, deslanoside, digitoxin, digoxin, digitalin and digitalis.

Neuromuscular blocking agents, include, for example, without limitation, atracurium mesylate, gallamine triethiodide, hexafluorenium bromide, metocurine iodide, pancuronium bromide, succinylcholine chloride (suxamethonium chloride), tubocurarine chloride, and vecuronium bromide.

Sedatives, include, for example, without limitation, amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride paraldehyde, pentobarbital, pentobarbital sodium, phenobarbital sodium, secobarbital sodium, talbutal, temazepam, and triazolam.

Local anesthetic agents, include, for example, without limitation, bupivacaine hydrochloride, chloroprocaine hydrochloride, etidocaine hydrochloride, lidocaine hydrochloride, mepivacaine hydrochloride, procaine hydrochloride, and tetracaine hydrochloride.

General anesthetic agents, include, for example, without limitation, droperidol, etomidate, fentanyl citrate with droperidol, ketamine hydrochloride, methohexital sodium, and thiopental sodium.

Radioactive particles or ions, include, for example, without limitation, strontium, rhenium, yttrium, technetium, and cobalt.

Any combination of one or more therapeutic agents or dugs can be used in the methods, compositions and kits provided herein. Such therapeutic agents or drugs can also be used in combination with anyone or more of the vascular permeability agents and/or labeled cells provided herein.

In some embodiments of the compositions, methods and kits provided herein, the therapeutic agent is also a vascular permeability agent.

Further information regarding additional therapeutic agents can be found in International Patent Application No. PCT/US10/54358, filed, Oct. 27, 2010, which is incorporated in its entirety by reference herein. For example, in some embodiments, a therapeutic agent can be administered to the damaged or diseased target tissue prior to (or concurrently or after) administration of the bi-functional composition. For example, in some embodiments, a therapeutic agent (e.g., a factor that reduces inflammation) can be administered to the injured damaged tissue within 2, 4, 6, 10, 12 or 20 hours, or about 1, about 2, about 3, about 4, about 5, about 6 or about 7 days of an injury, e.g., a myocardial infarction or contraction of an infection. In some embodiments, therapeutic agents are optionally labeled with magnetic particles to enhance their targeting.

In some embodiments, the bi-functional compositions provided herein are used in combination with an agent or other type of intervention that temporarily or permanently reduces blood flow to or through the target area (e.g., an embolic). In other embodiments, the methods provided herein can be used in combination with an agent or other type of intervention that lowers the heart rate and/or cardiac contractility (thereby reducing blood flow rate). In some embodiments, the agent is adenosine (e.g., 1 mg of adenosine within 1, 2, 5, 10, 15, 30, 45 or 60 min of cell delivery), verapamil, a beta-adrenergic blocker (e.g., propranolol, atenolol), a muscarinic agonist (e.g., methacholine) or combinations thereof. In other embodiments, cardiac contraction is suppressed with an agent that uncouples excitation and contraction, e.g., 2,3-butanedione-2-monoxime (BDM). In other embodiments, the delivery site at the target tissue is “sealed” with, for example, a fibrin glue (FO) (e.g., a solution of a thrombin/calcium chloride and a fibronectin/aprotinin mixed immediately before application). However, in some embodiments, no alterations (pharmacologic or physical) are made in order to affect the heart rate or contractility of a subject's heart (or other target tissue). In several embodiments, such additional agents can be used to counteract the effect of blood flow washing the delivered bi-functional compositions out of the target site. Thus, such embodiments improve cell retention or engraftment rates in the target tissue or organ. In some embodiments, administration of agents that slow ventricular rate can improve cell retention. In another embodiment, the therapeutic agents as used herein can be hydrogel such as fibrin glue. In some embodiments, co-administration of hydrogel such as fibrin glue also can improve retention by preventing cell washout.

### Diagnostic Imaging

In some embodiments, the compositions disclosed herein are also useful for monitoring the status of tissue (e.g., cells or organs) transplanted into a subject. In several embodiments, magnetic resonance imaging (MRI) is used to detect a signal based on the presence of the magnetic particles in a target tissue. By way of illustration only, the magnetic particles can lead to a marked decrease in the MRI parameter T2* and offer the possibility of non-invasive in vivo tracking of the composition. In addition, the coupling of one or more antibodies to the magnetic particles that are directed to activated immune cells enhance the ability to identify areas of a target tissue that are undergoing an immune response, which is suggestive of transplant rejection. The antibodies thus serve to localize the particles at the immune-reactive site, and the magnetic detection of the particles (e.g., by MRI) allows the determination of the existence, and also the severity of immune response in the tissue.

As discussed herein, in vivo and non-invasive techniques for monitoring the immune/rejection status of cardiovascular tissue are heretofore unknown. Use of the methods as disclosed herein, e.g., coupling of a specific antibody to the particle, and allowing the particle to ‘home’ to its target tissue molecularly (e.g., by antibody-antigen interaction) and/or supplementing the homing with an applied magnetic field, allow the specific localization of the compositions to target tissues. In the case of the heart, naked iron nanoparticles (as contrast agents) and MRI have been used for noninvasive detection of acute cardiac allograft rejection, but this approach heavily depends on the efficiency of endocytosis of injected iron particles by macrophages.

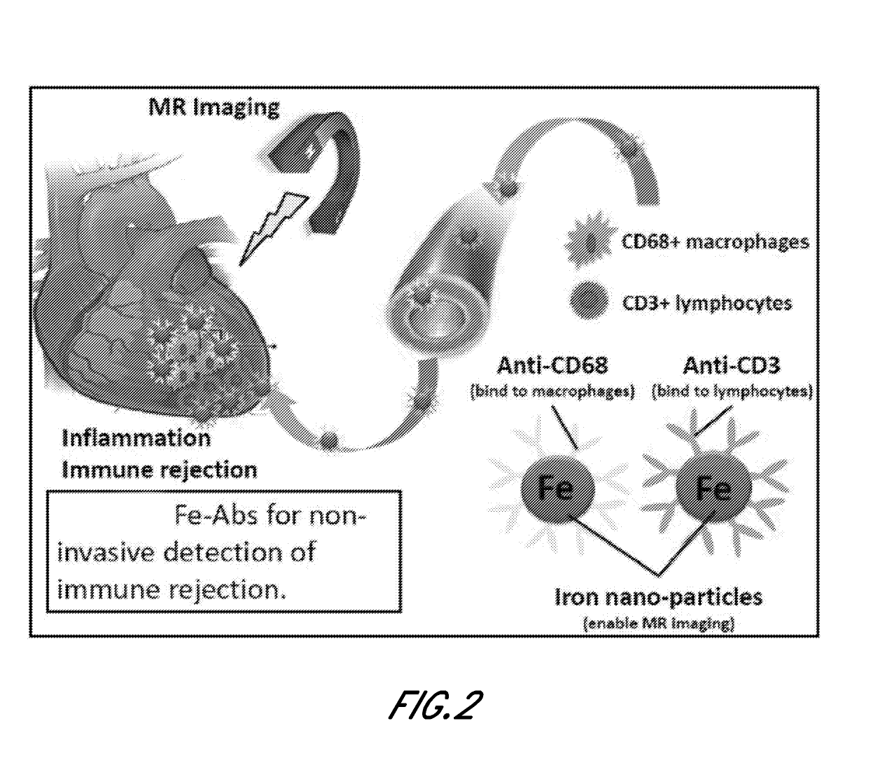

However, T lymphocytes, a major player in acute immune rejection, are not prone to endocytose iron particles. Thus, magnetic particles coupled to antibodies that are directed against T-lymphocytes, optionally used in conjunction with those directed to macrophages (see e.g., FIG. 2) advantageously generate a specific signal due to immune activated cells in the heart. Such a diagnostic method can be in place of, or in addition to more traditional methods (e.g., biopsy assessment of rejection status). Additionally, in several embodiments, other non-invasive techniques may be used to supplement MRI imaging (e.g., measurement of serum markers of rejection). Thus, the methods disclosed herein advantageously allow for non-invasive assessment of existence and severity of immune rejection post-transplant (in the heart or in other target organs expressing specific markers). These embodiments allow for the tailored treatment approach for each post-transplant patient based on the existence and/or severity of transplant rejection.

### Methods of Delivering Bi-functional Magnetic Particles

In several embodiments, the bi-functional magnetic compositions are delivered systemically to a recipient. In such embodiments, the composition can pass through the circulation of the recipient, capture a plurality of therapeutic cells, continue through the circulation, and interact with the markers expressed on the target tissue. This interaction, at least temporarily, immobilizes the composition at the target site, allowing for the therapeutic cell to provide its therapeutic effect. In some embodiments, delivery is systemic, but the site of administration is chosen in order to ensure that the composition passes through an area of the circulation that will first provide an opportunity for the antibodies on the particles to interact with the therapeutic cells prior to the target cell antibodies interacting with markers on the target tissue. In other words, such an approach allows the “loading” of the composition with therapeutic cells prior to the composition being directed to the target tissue. In other embodiments, the order in which interactions with the target tissue and the therapeutic cells do not adversely impact the overall therapeutic effect (e.g., the composition can first interact with markers on the target tissue, either with or without the aid of a magnetic field, and at a later time, the therapeutic cells, through natural circulation, will interact with the composition). In some embodiments, the compositions are delivered to a subject before, during or after a surgery. In some embodiments, the delivery takes place while the organ is actively functioning (e.g., while the heart is beating) rather than during a period of artificial inactivity (e.g., occlusion of a blood vessel to target the vascular wall). In other embodiments, the compositions are delivered to a tissue or organ using non-surgical methods, for example, either locally by direct injection into the selected tissues, to a remote site and allowed to passively circulate to the target site, or to a remote site and actively directed to the target site with a magnet. Such non-surgical delivery methods include, for example, infusion or intravascular (e.g., intravenous or intra-arterial), intramuscular, intraperitoneal, intrathecal, intradermal or subcutaneous administration.

In some embodiments, magnetic particle-labeled cells are administered with one or more therapeutic agents, either magnetized or unmagnetized, to a target tissue or organ. Such therapeutic agents include, for example, antineoplastic agents, angiogenic factors, or anti-angiogenic factors, immuno-suppressants, or antiproliferatives (anti-restenosis agents), embryonic factors, fibroblast growth factors, hematopoietic growth factors, transcription factors, kinase inhibitors or adenosine. Other non-limiting examples of therapeutic agents are provided elsewhere herein. Such therapeutic agents can be administered before, concurrently, or after the compositions, depending on the embodiment. In still additional embodiments, additional therapeutic cells (e.g., exogenous cells) are administered to further enhance the therapeutic effects achieved.

### Compositions and Methods of Using Bi-functional Magnetic Compositions to Treat Heart Diseases

In some embodiments, the compositions and methods provided herein employ the bi-functional magnetic compositions and are useful for treating an injured cardiac tissue in a subject by reducing or ameliorating the progression, severity or duration of a cardiac tissue injury or a symptom thereof. In some embodiments, treatment preserves the injured cardiac tissue and function thereof, such as by preserving or reducing cell apoptosis, or by reducing cell inflammation. In other embodiments, treatment regenerates cardiac tissue, e.g., cardiac muscle and/or cardiac vasculature. In some embodiments, treatment activates or enhances cell proliferation or cell migration. In some embodiments, treatment increases blood flow to the injured tissue. In some embodiments, treatment increases myocardial perfusion. In some embodiments, treatment regenerates new cardiac tissue. In other embodiments, treatment increases cardiac muscle mass. In several embodiments, two or more of the above-mentioned functional or physiological parameters are improved.

In some embodiments, treatment improves global cardiac function. In some embodiments, improvements in global cardiac function are measured by, for example, stroke volume, ejection fraction, cardiac contractility and/or cardiac output using any method known in the art. In some embodiments, improving global cardiac function comprises increasing cardiac output. In some embodiments, improving global cardiac function comprises increasing ejection fraction (e.g., the fraction of blood pumped out of a ventricle with each heart beat) by at least an absolute range of about 5% to about 25%, about 5% to about 10%, about 5% to about 15%; about 5% to about 20%, about 10% to about 15%, about 10% to about 20%, about 10% to about 25%, about 15% to about 20%, about 15% to about 25%, about 20% to about 25%, and overlapping ranges thereof. Ejection fraction can be assessed by a number of methods known in the art. In some embodiments, the ejection fraction is determined by echocardiography, cardiac MRI, fast scan cardiac computed axial tomography imaging, or ventriculography. In some magnetic particle-embodiments, the ejection fraction is assessed by echocardiography.

In other embodiments, treatment improves regional cardiac function. In some embodiments, improvements in regional cardiac function are measured by wall thickening, wall motion, myocardial mass, segmental shortening, ventricular remodeling, new muscle formation, the amount of cardiac cell proliferation and programmed cell death (or their relative proportions), angiogenesis and/or the size of fibrous and infarct tissue. In some embodiments, improving regional cardiac function comprises increasing heart pumping. In some embodiments, cardiac cell proliferation is assessed by the increase in the nuclei or DNA synthesis of cardiac cells, cell cycle activities or cytokinesis. In some embodiments, programmed cell death is measured by TUNEL assay that detects DNA fragmentation. In some embodiments, programmed cell death can be assessed by measuring the expression levels of one or more genes or proteins known to be involved in the apoptotic cascade (e.g., caspases). In some embodiments, angiogenesis is detected by the increase in arteriolar and/or capillary densities. In some embodiments, cardiac function before and after treatments are assessed by echocardiography (e.g., transthoracic echocardiogram, transesophageal echocardiogram or 3D echocardiography), cardiac catheterization, magnetic resonance imaging (MRI), sonomicrometry or histological techniques. Techniques in assessing cardiac function can also be performed using established methods and procedures known in the art.

In some embodiments, improving global cardiac function comprises increasing ejection fraction by about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%. In several embodiments ejection fraction is increased by about 2-fold, about 5-fold, or about 10-fold.

For example, in some embodiments, a patient having a tissue injury, such as a myocardial infarction, will have an ejection fraction of between about 40% to about 55% that will improve to about 65% or greater after being subjected to a method provided herein. In some embodiments, improvements in one or more of the parameters discussed herein may or may not be associated with improvements in other parameters. For example, increases in ejection fraction, in some embodiments, may be detected despite minimal changes in cell proliferation or myocardial mass.

In several embodiments, dual targeting of the therapeutic compositions with the combination of magnetic fields and antibodies results in a prolonged (and/or synergistically increased) therapeutic effect in the target tissue (for example, as compared to magnetic targeting alone or molecular targeting alone). As discussed above, in some embodiments, magnetic targeting enables the localization of cells to a target tissue of interest (with more specific targeting to particular regions or subtypes of tissue based on the antibodies). However, in some cases, magnetic targeting alone reaches a “ceiling” of therapeutic efficacy related to the number of magnetic cells delivered to the target tissue. Magnetic targeting in conjunction with antibody targeting (in particular in embodiments wherein the antibodies were specifically selected for their characteristics of interaction with their antigen) can increase the duration and/or efficacy of the composition. For example, in several embodiments, antibodies populations are selected to have particular binding characteristics with their antigen. In several embodiments, the antibody population that interacts with an epitope on a diseased or damaged tissue is selected such that the interaction is high affinity, high avidity, or a combination thereof, with the end result being that the antibody binds the epitope for an extended duration of time (as compared to an interaction with lesser affinity and/or avidity). Coupled with this specifically chosen antibody population to interact with the target tissue, an antibody population for interaction with therapeutic cells is chosen, in certain embodiments, to have a reduced affinity, avidity, or both. As such, there is an increase in the turnover (e.g., binding of cells to the antibody and subsequent release) of therapeutic cells at the target site. In several embodiments, the release of the therapeutic cells from the antibody is due to their integration into the target tissue (e.g., resulting in direct repair/regeneration of the tissue). In some embodiments, direct incorporation of the cells is not necessary for a therapeutic effect to be realized. In several embodiments, one or more paracrine signals (e.g., growth factors, signaling molecules etc.) are released from the recruited therapeutic cells and positively impact the damaged or diseased target tissue. The release of a first “batch” of therapeutic cells and binding of a subsequent “batch” of therapeutic cells results in an effective delivery (over time) of a greater number of therapeutic cells, and, in turn, a greater therapeutic effect.

In some embodiments, cardiac tissue subjected to the methods provided herein has been injured, for example, due to ischemia, infarction, reperfusion or occlusion. In some embodiments, the cardiac tissue is focally injured or diseased while in other embodiments, the tissue is diffusely injured or diseased. In some embodiments, the cardiac tissue is injured as a result of acute stress, for example, acute heart failure. In other embodiments, the cardiac tissue is injured as a result of chronic stress or injury/disease, for example, chronic heart failure, systemic hypertension, pulmonary hypertension, valve dysfunction, congestive heart failure, or atheromatous disorders of blood vessels (e.g., coronary artery disease). In some embodiments, the injured cardiac tissue is in the epicardium, endocardium and/or myocardium. In some embodiments, the subject is a mammal, such as a non-primate. In some embodiments, the subject is a human. In one embodiment, the subject is a human with acute heart failure or chronic heart failure.

### EXAMPLES

### Example 1

### Targeted Bi-functional Magnetic Compositions for the Treatment of Cardiovascular Diseases

The present studies relate to the ability to couple antibodies to magnetic particles, capture therapeutic cells with one portion of the antibodies, and foster interaction (enhanced via magnetic field generation) between a second portion of the antibodies and a damaged or diseased target tissue. The embodiments disclosed herein combine the advantages of molecular targeting and magnetic targeting to generate synergistic therapeutic effects. Also, as discussed below, several embodiments will provide more accurate and precise diagnostic applications.

Without being bound by theory, it is believed that boosting the body's endogenous stem cell recruitment will not only fulfill, but surpass, the treatment effects as a result of exogenous stem cells. In conjunction with magnetic targeting, synergistic therapeutic effects are achieved, in several embodiments.

Iron Nanoparticles can be Tagged with Antibodies

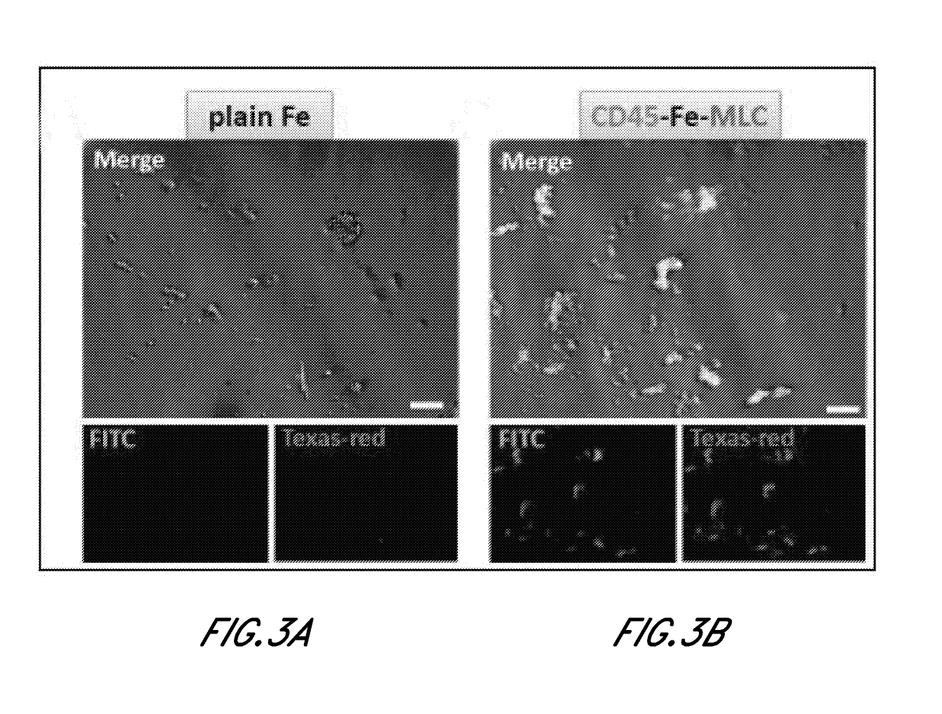

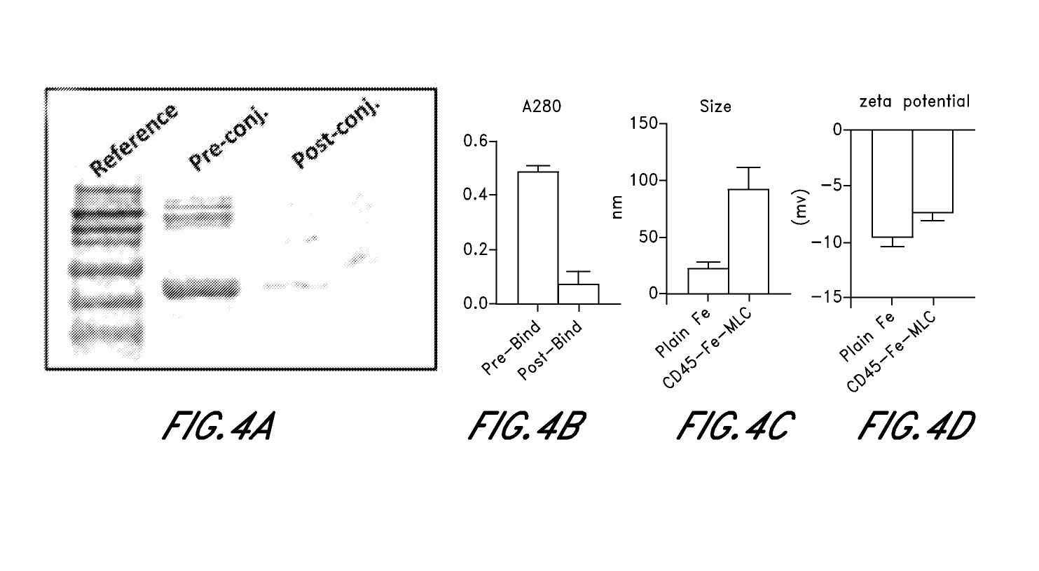

As a threshold issue, experiments were performed to demonstrate magnetic particles could be coupled to antibodies. In order to provide a reduced barrier to therapeutic applications in the clinic, an iron nanoparticle that has gained FDA approval. FERAHEME® was chosen as the magnetic particle. FERAHEME® is approved for the treatment of iron deficiency anemia in adult patients with chronic kidney disease. Advantageously, however, the carboxyl (COOH) groups on FERAHEME® particles enable covalent coupling of antibodies to the magnetic particle by activating the carboxyl groups with water-soluble carbodiimide (e.g. EDAC reagent). The following reagents were mixed (though, depending on the embodiment, different amounts, concentrations, or equivalent reagents are used in other embodiments), : 100 μL of anti-CD45 (1 mg/mL); 100 μL of anti-myosin light chain (1 mg/mL); 40 μL of EDAC reagent; 40 μL of FERAHEME®; 340 μL of Protein Coupling Buffer. The solution was incubated at 37° C. for 4 hours. Longer or shorter times may also be used, depending on the embodiment and the degree (e.g., concentration) of conjugation desired for that particular embodiment). Ultra centrifugation was performed to collect the conjugated particles and the supernatant was removed. FERAHEME® particles were conjugated with mouse anti-MLC (antibody 1) and rabbit anti-CD45 (antibody 2) antibodies via incubation with the EDAC reagent, followed by incubation with the antibodies. These antibodies were chosen as proof of principle, and depending on the embodiment, any number of combinations of different antibodies may be chosen and be within the scope of the present disclosure (for example, anti-CD34 antibodies, anti-c-kit antibodies, cancer cell-specific antibodies, or combinations thereof etc.) Anti-rabbit (FITC) and anti-mouse (Texas-red) secondary antibodies were used to detect the primary antibodies. As a negative control, plain Fe underwent the same secondary antibody staining experiment. Successful conjugation was confirmed as the presence of FITC and Texas-red fluorescence (FIG. 3A). Plain iron beads showed no fluorescence (FIG. 3B). Additional data confirming the coupling are shown in FIGS. 4A-4D. FIG. 4A shows the shift in molecular weight of the antibodies (as measured by SDS-PAGE) after coupling to the microparticles. FIG. 4B shows the reduction in protein concentration in the post-coupling antibody reagent (e.g., demonstrating the “loss” of protein as the antibodies are coupled to the microparticle). FIG. 4C shows that the size of the magnetic particles increases post-coupling). FIG. 4D shows that the zeta potential of magnetic particles coupled to antibodies is unchanged, suggesting that the bi-functional compositions can be administered via intravenous routes (as with uncoupled FERAHEME®). Taken together, these data demonstrate that antibodies can successfully be coupled to magnetic particles, thereby indicating that the compositions of the embodiments disclosed herein can be successfully manufactured.

### Bi-Functional Magnetic Particles Bind to Injured Cardiomyocytes In Vitro

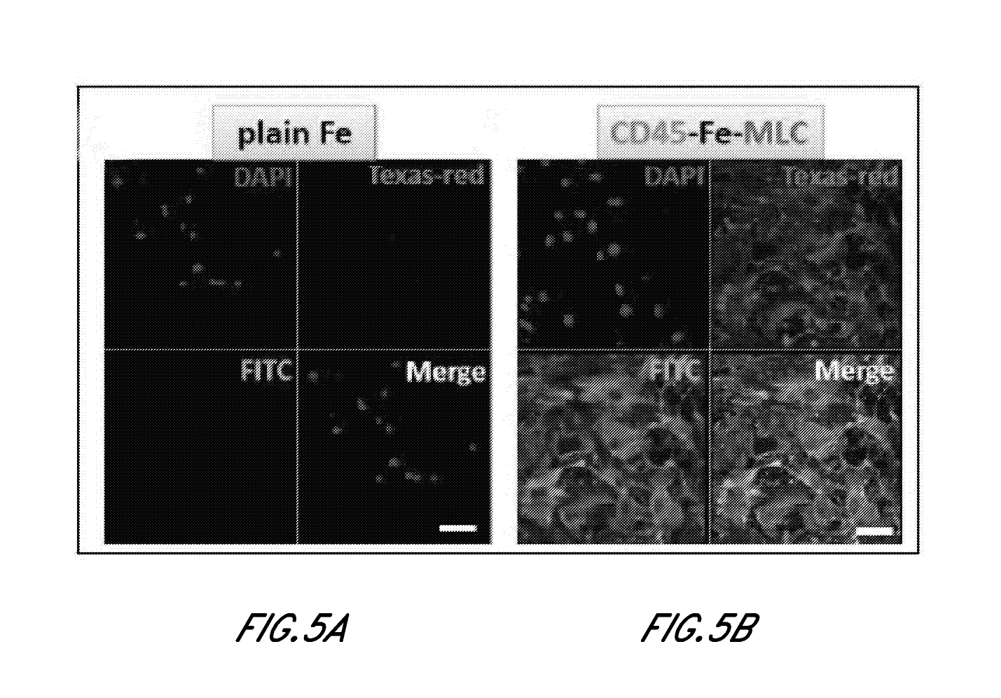

After demonstrating that antibodies could be coupled to magnetic particles, experiments were performed to demonstrate that antibody-coupled magnetic particles could bind to specific target tissues according to the antibodies chosen. Neonatal rat cardiomyocytes (NRCMs) were permeabilized with CYTOPERM/CYTOFIX (Becton Dickenson) to produce membrane injury mimicking that seen after myocardial infarction. To target the composition to injured myocytes, a target tissue antibody directed against myosin light chain (MLC) was coupled to the magnetic particles. MLC is an epitope exposed only when the membrane of a cardiac cell is disrupted (e.g. after infarction). The cells were incubated with either plain FERAHEME® particles or antibody-linked FERAHEME® particles (CD45-Fe-MLC). Cells were subsequently stained with FITC- or Texas red-conjugated secondary antibodies. As shown in FIG. 5A, iron particles that are not coupled to antibodies show no signal after secondary antibody staining (5A upper right and lower left) and do not bind to injured NRCMs (no difference between nuclei shown in 5A upper left and 5A lower right (merged channels)). In contrast, bi-functional compositions of magnetic particles coupled to anti-CD45 and anti-MLC antibodies (CD45-Fe-MLC) efficiently bind to injured NRCMs (FIG. 5B). Signals in FIG. 5B upper right and lower left reaffirm that the primary CD45 and MLC antibodies were coupled to the particles. The merged figure in 5B lower right confirms that the CD45 and MLC antibodies co-localized with the nuclei of the NRCMs. These data thus indicate that specific binding of the bi-functional compositions disclosed herein to a target tissue can be achieved. Again, CD45 and MLC were antibodies chosen as examples that demonstrate the functionality of the embodiments disclosed herein, and, depending on the embodiment, other antibodies, and other combinations of antibodies (e.g., CD34 and MLC, c-kit and MLC, CD34 and a cancer marker, or CD34 and another antibody, etc.) are used in other embodiments.

### Bi-Functional Magnetic Particles Successfully Deliver Bone Marrow Stem Cells to Injured Cardiomyocytes

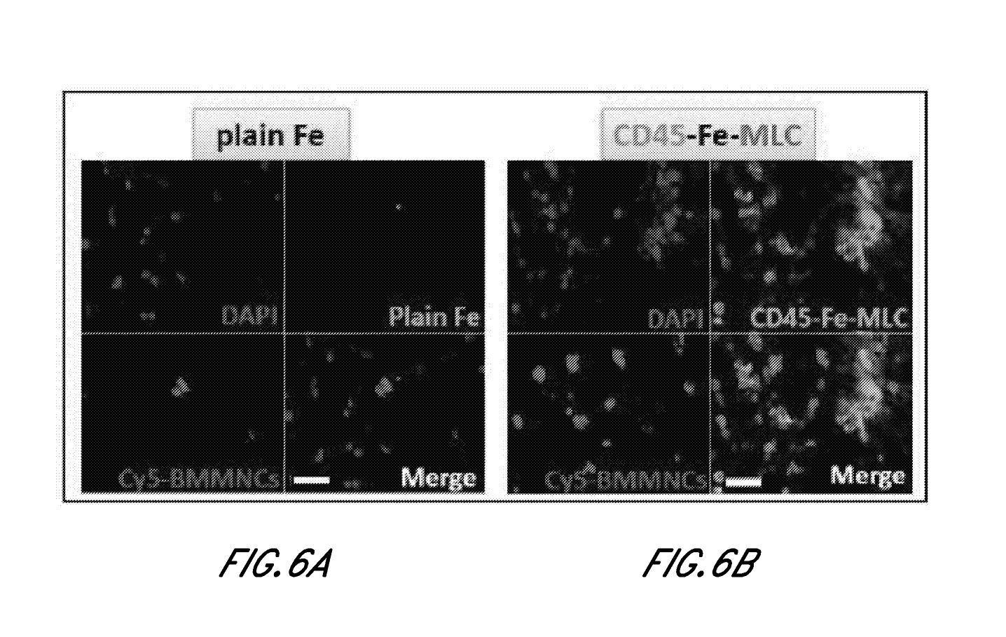

Building on the experiments above, the present study set out to determine whether bi-functional magnetic particles (e.g., CD45-Fe-MLC) could successfully link stem cells with cells of a target tissue. Used as examples of the embodiments disclosed herein, bone marrow stem cells were chosen as the example for a therapeutic cell population and permeablized NRCM as being representative of injured cardiomyocytes. Injured NRCMs were incubated with plain iron particles or CD45-Fe-MLC particles for one hour and then further incubated with CD45+ bone marrow mononuclear cells (BMMNCs), labeled with far-red dye DiD (Invitrogen). After incubation, the culture was washed with PBS to remove un-attached BMMNCs before analysis. As shown by the fluorescent images in FIG. 6A, plain iron microparticles did not colocalize with BMMNCs and/or NRCMs. In contrast, BMMNCs (magenta) did show substantial colocalization with the CD45 and MLC antibodies (see FIG. 6B lower right). These results indicate that iron particles bound to a targeting antibody and an antibody designed to capture a therapeutic cell are functional at both “ends”. In other words, the bi-functional nature of the compositions described herein can successfully be achieved. As such, the bi-functional compositions (such as CD45-Fe-MLC) can be used, in several embodiments, to enhance the endogenous recruitment of stem cells to injured cardiomyocytes in vivo. In some embodiments, the targeting antibody and/or the therapeutic cell antibody may be different than those tested in this proof of concept example. For instance, in several embodiments, cardiac targeting markers are coupled to magnetic particles along with a broader stem cell capturing antibody, such as c-kit. This is advantageous, in several embodiments, as c-kit+ stem cells have been shown effective in heart regeneration through both direct regeneration and paracrine effects. In several embodiments, in place of c-kit antibodies, CD34 antibodies could also be used. In still additional embodiments, cardiac stem cell specific markers are used.

### Injected Bi-Functional Magnetic Particles Successfully Target Injured Myocardium

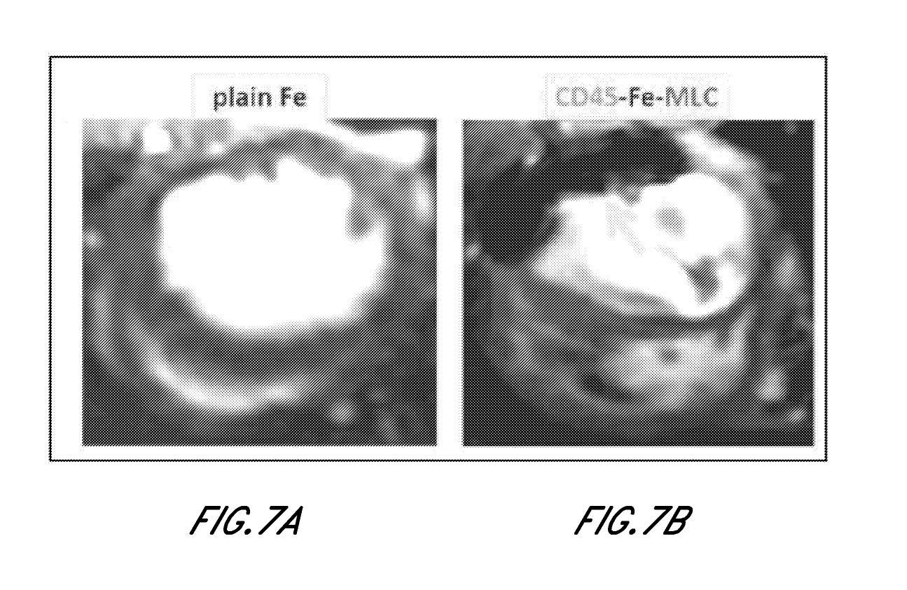

In order to demonstrate the ability of the antibody coupled magnetic particles to home to a specific target tissue, myocardial infarction was induced in Wistar Kyoto rats with established ischemia/reperfusion methods. Three days later, plain iron particles or CD45-Fe-MLC (as an example of embodiments disclosed herein) particles were delivered. Cardiac MRI scanning was performed after an additional days. While little signal could be detected via MRI in the heart of animals receiving plain iron particles (FIG. 7A), large areas of iron particles (representing the CD45-Fe-MLC composition) could be visualized (as a black signal void) in the infarcted area (FIG. 7B).

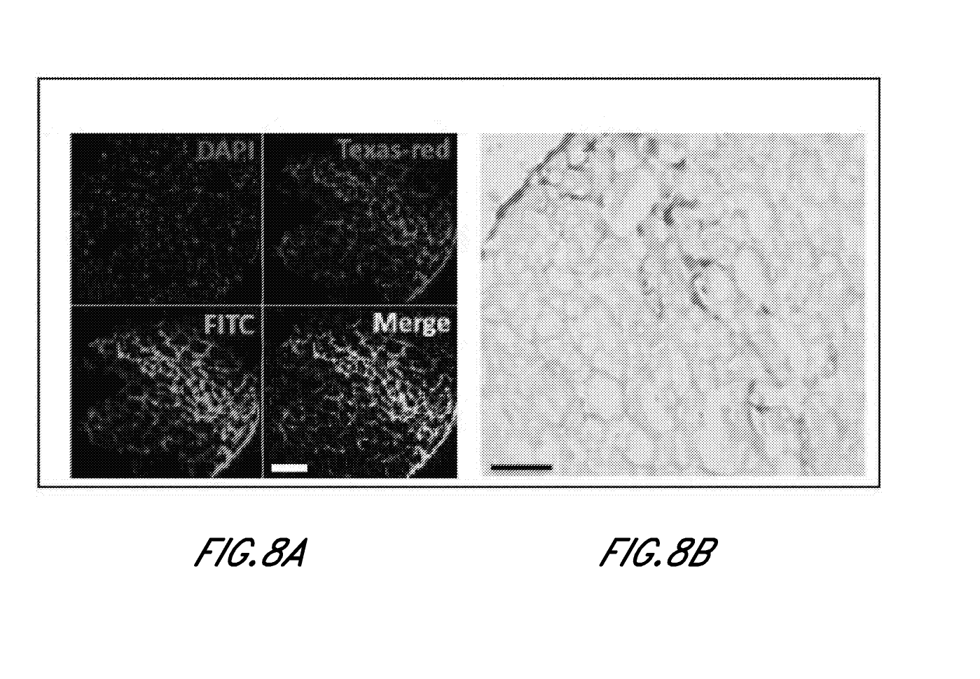

After MRI analysis, the rats were sacrificed and the hearts were excised, cryo-sectioned and then stained with FITC- or Texas red-conjugated secondary antibodies. Confocal microscopy revealed the presence of specific FITC and Texas red fluorescence (FIG. 8A, lower left and upper right panels, respectively), indicating the existence of CD45-Fe-MLC particles in the infarcted area. The fluorescence pattern was consistent with Prussian Blue staining, which is specific to iron, and therefore represents detection of the iron particles (FIG. 8B). This correlation demonstrates that not only are the iron particles present in the target tissue, but they are present in a distribution that indicates that they are still coupled to functional antibodies. These data indicate that, in several embodiments, the bi-functional compositions not only have the capacity to migrate to a desired target tissue, but that they also can successfully capture therapeutic cells at the target site. Thus, in some embodiments, the bi-functional compositions successfully serve as a linker or bridge between the damaged target cells and the therapeutic cells, thereby allowing repair and/or regeneration of the damaged target cells.

### Conjugation of Antibodies to Nanoparticles

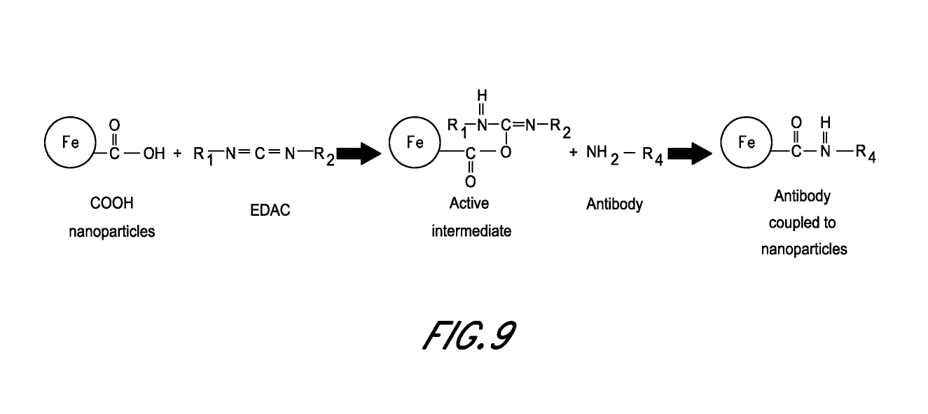

As discussed above FERAHEME® nanoparticles, also known as ferumoxytol (AMI-228), were recently (Jun. 30, 2009) FDA-approved for treatment of iron deficiency anemia. FERAHEME® is a member of the class of monodispered nano-sized ultra-small superparamagnetic iron oxides (USPIO). It has a molecular weight of 73 lkD and a hydrodynamic diameter of about 30 nm, and a chemical formula of Fe₅₈₇₄O₈₇₅₂-C₁₁₇₁₉H₁₈₆₈₂O₉₉₃₃Na₄₁₄₂. Capitalizing on the carboxylated polymer coating, in several embodiments, covalent coupling of antibodies is achieved by activating the carboxyl groups. In several embodiments, the groups are activated with water-soluble carbodiimide (e.g. EDAC reagent). The EDAC reagent reacts with the carboxyl group to create an active ester that is reactive toward primary amines on the antibodies of interest (FIG. 9). Other reaction schemes are used in other embodiments, depending both upon the antibody used and/or on the magnetic particles and any coating or treatment the chosen particles have. Advantageously, FERHEME® have very little free iron present, allowing large amounts to safely be administered (510 mg of FERAHEME has been administered safely in as little as 17 sec for a rate of 30 mg/sec). Time and temperature of conjugation reactions are varied, depending on the embodiment. Concentrations of FERAHEME®, EDAC reagent, and the antibodies are also varied depending on the embodiment. Conditions for optimizing, depending on the antibodies chosen, are evaluated, in several embodiments, according to the methods described above (e.g., immunohistochemistry, SDS-PAGE, A₂₈₀, zeta potential, etc.)

Use of Bi-functional c-kit-Fe-MLC or CD34-Fe-MLC to Target Injured Cardiomyocytes

To demonstrate that different antibodies that target therapeutic cells can be used, the contemplated experiments have been designed. Neonatal rat cardiomyocytes (NRCMs) will be derived according to standard established protocols. NRCMs will be cultured on chamber slides and treated with permeablization agent (Becton Dickenson Cytoperm/Cytofix) for 10 min to allow expression of MLC on the surface, mimicking cardiomyocyte injury in vivo (after myocardial infarction, the membrane of cardiomyocytes is disrupted to release myosin light chain (MLC) proteins which is normally not expressed on the surface of healthy cardiomyocytes). Three experimental phases will be performed:, (i) Phase 1—ckit-Fe-MLC (or CD34-Fe-MLC) and plain Fe (no antibody linked) particles will be incubated with the permeablized NRCMs for one hour at 37° C. Binding efficiency will be evaluated by secondary antibody staining and fluorescent microscopy as described above; (ii) Phase 2—orbital shaking will be introduced into the incubation reaction to mimic the dynamic fluidic environment in the heart and a permanent magnet will be mounted on the bottom of NRCM culture to physically attract the ckit-Fe-MLC (or CD34-Fe-MLC) particles; and (iii) Phase 3—DiD-labeled and ckit-expressing (or cD-34-expressing) bone marrow stem cells (BMSCs) will be introduced into the culture during the 1 hour incubation time with engagement of BMSCs with NRCMs to be assessed by fluorescent microscopy as described above. These experiments will indicated whether ckit-Fe-MLC (or CD34-Fe-MLC) can specifically target injured cardiomyocytes and engage them with ckit+BMSCs.

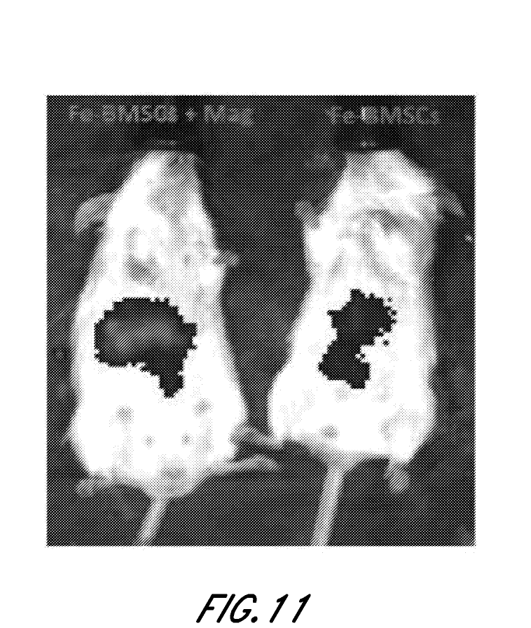

In vivo studies will test if ckit-Fe-MLC (or CD34-Fe-MLC) can capture circulating ckit-expressing (or CD-34 expressing) BMSCs in vivo and direct them to the injured myocardium. Exogenous syngeneic BMSCs will be systemically delivered into recipient rats to mimic “circulating endogenous BMSCs”. BMSCs will be derived from syngeneic Wistar Kyoto rats by tibia bone isolation and density centrifugation. Flow cytometry analysis will be performed to confirm the ckit expression (or CD34 expression) in BMSCs prior to administration. BMSCs will be labeled with far-red dye DiD to allow fluorescent imaging and histological detection. Myocardial infarction will be created by 3 hours of ischemia (LAD ligation) followed by vessel reperfusion for 3 days. Prior results have indicated that this procedure is sufficient to create a reasonable infarct (FIGS. 9A-9B), as confirmed by cardiac MRI and Masson's trichrome staining. The in vivo experiment will also have 3 phases, (i) Phase 1—2 million BMSCs will be intravenously (i.v.) injected through the tail vein, together with plain Fe or ckit-Fe-MLC (or CD34-Fe-MLC). The accumulation of BMSCs in heart will be checked by live fluorescent imaging and heart histology; (ii) Phase 2—dose optimization of magnetic particles will be performed. A dose of 7 mg Fe/kg of rat body weight, based on established human FERAHEME® doses will be used as the initial point from which doses will be escalated to determine if enrichment of BMSCs can be enhanced by higher doses of ckit-Fe-MLC (or CD34-Fe-MLC); and (iii) Phase 3—the effect of magnetic targeting to enhance the homing of BMSCs will be evaluated. A circular rare earth magnet will be mounted on the thoracic region of the rats during BMSC and ckit-Fe-MLC (or CD34-Fe-MLC) infusion and left there for one of 3 different time periods (1 hour, 24 hour and 48 hour). Prior results indicated that magnetic targeting is capable of enriching systemic delivery of BMSCs to the liver (Figure. 11). In order to reduce the possible confounding data that could be generated by general IgG binding, an isotype IgG-Fe control will be included, in some embodiments. To ensure accurate quantitative methods to determine BMSC counts in the heart, sex mismatch PCR will be used, in some embodiments (delivery of male BMSCs to female recipients to enable qPCR detection of SRY gene located in the male Y chromosome).

Assessment of the Toxicity of Fe-Abs in Cultured Cells and Naïve Animals

While prior data has indicated that FERAHEME® labeling did not affect the viability and proliferation of cardiac stem cells (FIGS. 12A-12B) and unconjugated FERAHEME® has shown an excellent safety profile in preclinical animal studies and clinical trials, the potential toxicity of FERAHEME® antibody conjugates (Fe-Abs) is largely unknown. Thus, the contemplated studies will be performed to determine the toxicity (or other side effects) of antibody-conjugated magnetic particles. The in vitro toxicity of Fe-Abs on cultured stem cells (e.g. ckit+BMMNCs) and cardiomyocytes will be studied by comparing cell viability, reactive oxygen species (ROS) generation, and differentiation potential with that of naïve cells. In addition, in vivo toxicity studies will be performed by i.v. injection of Fe-Abs up to 5 mg Fe-Abs/kg/day for 12 weeks in healthy rats (cumulative exposure approximately 5 times the anticipated exposure of a human therapeutic course of FERHEME on mg/m² basis) and assessing the following parameters: mortality, body weight loss, food consumption, increases in pigmentation intensity, red blood cell counts, hemoglobin and serum iron/transferrin/ferritin, liver and spleen weight, liver function, immunogenicity to the antibodies, and the biodistribution of Fe-Abs in various organs by MRI and histology. Potential toxicity due to magnetic targeting will also be evaluated.

### Cells can be Captured and Magnetically Targeted

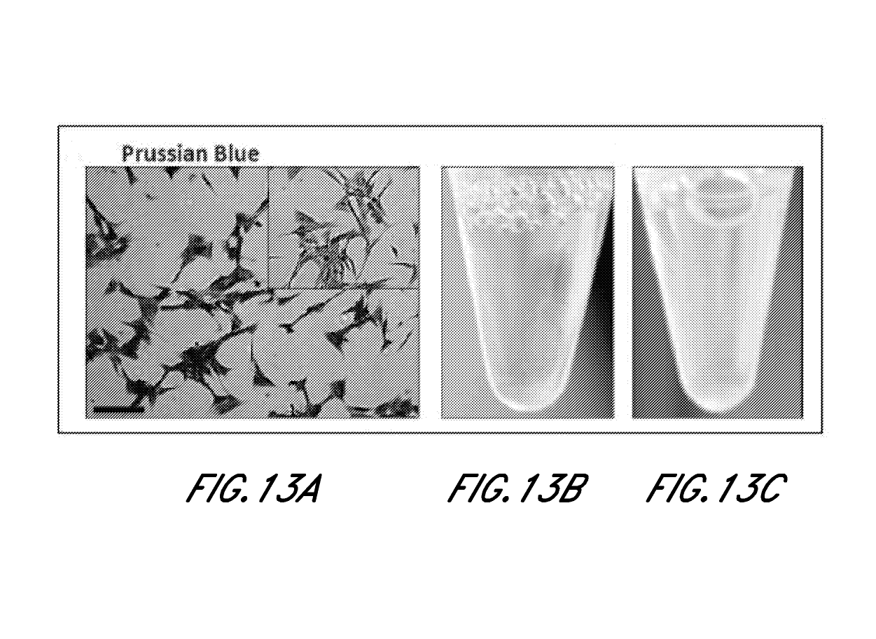

An additional issue that faces therapies with bi-functional compositions as described herein is whether cells can be captured and magnetically targeted. In other words, is the capture/linking of stem cells with antibodies coupled to magnetic particles strong enough to execute the idea of magnetic targeting. The following data indicates that such targeting is feasible. Cardiac stem cells were tagged with Fe-Abs, indicated by Prussian blue staining (FIG. 13A). A locally-placed magnet quickly attracted the Fe-Ab-tagged cells (FIG. 13B), but not the control untagged cells (FIG. 13C), to the tube wall. While the experiments described above indicate that, in some embodiments, antibody targeting alone may be sufficient to target a therapeutically effective amount of endogenous stem cells to the target tissue (e.g., injured myocardium) and exhibit sizable functional benefit, however, in several embodiments, magnetic targeting provides a synergistic effect that allows delivery of a greater number (or greater therapeutic effect) of cells as compared to molecular or magnetic targeting alone. Magnetic targeting systems may include, among other options, a high field gradient targeting systems utilizing the combination of a uniform magnetic field created by a MRI machine and an electromagnet pair.

### In Vivo Effects of Bi-Functional Magnetic Compositions

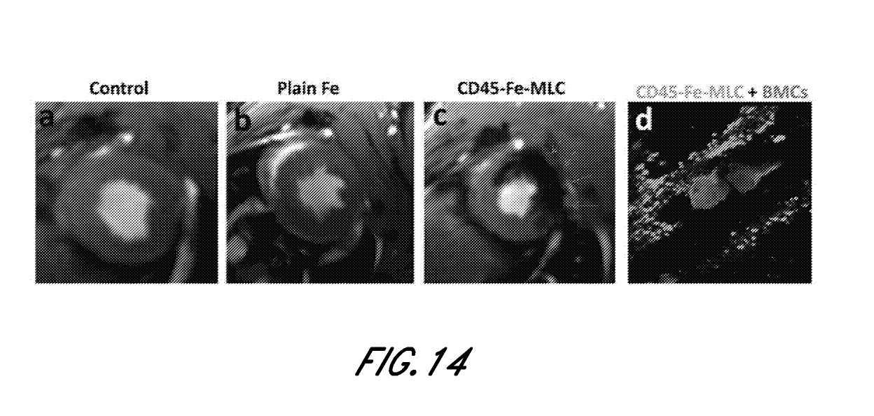

The present experiment was designed to replicate a therapeutic treatment of a subject using bi-functional magnetic particles coupled to target tissue antibodies and therapeutic cell antibodies. The CD45-FE-MLC composition was prepared and administered to rats with myocardial infarction as described above. As shown in FIG. 14B, the delivery of un-coupled (plain) iron particles did not yield any identifiable signal in the cardiac tissue by MRI. In contrast, as shown in FIG. 14C, the CD45-Fe-MLC composition specifically tagged injured myocardium (as detected by cardiac MRI). This was confirmed by immunohistochemistry. Moreover, the composition that was specifically targeted to the injured myocardium was able to successfully capture (and thus hold in a therapeutically relevant position) circulating BMCs (see FIG. 14D). These in vivo data demonstrate that the compositions and methods disclosed herein, in several embodiments, are capable of the targeted delivery of specific stem cells to specific target tissues, such as those injured or damaged to disease. In some embodiments, the target tissue is cardiac tissue (such as injured post-infarction cardiac tissue), while in some embodiments, other target tissues are treated. In some embodiments, bone marrow stem cells are used (or another widely circulating or liberated stem cell variety), while in other embodiments, endogenous stem cells related to the target tissue are captured and delivered to the target tissue (e.g., cardiac stem cells delivered to damaged cardiac tissues). In several embodiments, these methods lead to the repair and/or regeneration of target tissue such that functional and/or anatomical improvements are realized.

### Example 2

### Use of Targeted Nanomaterials for Diagnosis of Cardiac Immune Status

As discussed above, another embodiment in which the bi-functional compositions disclosed herein are used in is the non-invasive diagnostic testing of immune status of a particular tissue. Naked iron nanoparticles (as contrast agents) and MRI have been used for noninvasive detection of acute cardiac allograft rejection, but this approach heavily depends on the efficiency of endocytosis of injected iron particles by macrophages. However, T lymphocytes, a major player in acute immune rejection, are not prone to endocytose iron particles. Bi-functional particles that capture T lymphocytes and detect macrophages may, in several embodiments, create a specific MRI signal diagnostic for rejection. To this end, the following contemplated experiments have been designed.

### Capability of Fe-CD68 and Fe-CD3 to Target Immune Cells and Characterize the MRI Properties of Fe-Abs-Tagged Cells

Rat macrophages and lymphocytes will be isolated from Wistar Kyoto rats and cultured according to established methods. The expression of CD68 (macrophages) and CD3 (T-lymphocytes) will be confirmed by flow cytometry. Anti-CD68 and anti-CD3 antibodies will be coupled to FERAHEME® particles. Binding of Fe-CD68 and Fe-CD3 particles to macrophages and lymphocytes, respectively, in both static and dynamic conditions will be tested in vitro, similar to that described above. Binding efficiency will be evaluated by immune-staining and flow cytometry analysis.

In order to more clearly characterize the MRI signals (thereby allowing, in several embodiments, quantitative measurement of magnetic particles, and thus macrophages and T-lymphocytes) variant doses of FeAbs-tagged macrophages and lymphocytes will be locally injected into freshly isolated rat hearts and MRI will be performed. An index between the MRI signal and the amount of macrophages or lymphocytes in the heart will be generated by correlating the MRI signal attenuation with the actual cell amount. Hearts injected with naked macrophages and lymphocytes will be scanned as negative controls.

### Diagnostic Potential of Bi-functional Magnetic Compositions for Noninvasive Detection of Immune Reaction

A heterotopic heart transplantation model will be used in which Brown Norway (BN) and Wistar Kyoto (WK) rats will undergo abdominal heterotopic heart transplantation (AHT). These two strains of rats are widely used for acute rejection studies because of their strong immunogenic responses. Briefly rats will be anesthetized with pentobarbital (50 mg/kg IP), intubated, and ventilated. Donor rats will be heparinized (1000 U/kg IV), and the anterior rib cage opened to expose the heart. The inferior vena cava (IVC), superior vena cava, and pulmonary veins will be ligated and divided, the great vessels transected, and the explanted heart will be immersed in 4° C. saline. In recipient rats, a midline abdominal incision will be made, the donor aorta will be anastomosed to the recipient abdominal aorta, and the donor pulmonary artery will be anastomosed to the recipient abdominal IVC. The heart will be reperfused and the abdomen will be closed, and the rats will be allowed to recover. Acute rejection normally happens in days. Animals will be sacrificed at Day 3, Day 7, and Day 15 after AHT and heart histology will be performed. Hearts will be evaluated for presence of clusters of CD68+ macrophages and CD3+T lymphocytes.

Once the transplant model is established, Fe-Abs will be tested to determine if they can actively target immune rejection zones and create specific signals for detection by cardiac MRI. At Day 3, Day 7 and Day 15 after heart transplantation, the rats will receive i.v. infusion of either 1) PBS control; 2) plain Fe; 3) commercial contrast agent (e.g. gadolinium); 4) Fe-CD68; 5) Fe-CD3; 6) Combination of Fe-CD68 and Fe-CD3. Two hours later, the rats will be subject to cardiac MRI. Subpopulations of rats from each treatment group will be sacrificed after MRI and heart histology will be performed. Acute immune rejection zones will be identified by staining the sections with CD68, CD3, and TNF-alpha antibodies. H&E staining will be performed and the rejection grade will be scored by experienced pathologists. A correlation will be made between the histology-detected rejection patterns and the MRI images to determine whether Fe-CD68 and Fe-CD3 (or the combination) are superior to plain Fe and commercial contrast agents to detect immune rejection zones. As a safety endpoint, it will also be determined if the administration of Fe-Abs exacerbates the inflammation and immune reaction in the post-AHT heart. The total number of immune cells in the hearts from the control and Fe-Abs groups will be compared. Also, inflammatory cytokines (e.g., TNF-alpha, IFN-gamma, IL1-beta) in the serum will be measured. The dose of administered Fe-Abs will be optimized for imaging quality and the in vivo distribution and degradation of Fe-Abs will be monitored by MRI.

Although the embodiments of the inventions have been disclosed in the context of a certain preferred embodiments and examples, it will be understood by those skilled in the art that the present inventions extend beyond the specifically disclosed embodiments to other alternative embodiments and/or uses of the inventions and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. For all of the embodiments described herein the steps of the methods need not be performed sequentially. Thus, it is intended that the scope of the present inventions herein disclosed should not be limited by the particular disclosed embodiments described above.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

1. (canceled)

2. A method for treating damaged or diseased cardiac tissue comprising:
administering to a subject having damaged or diseased cardiac tissue a composition comprising:
magnetic particles coupled to a first population of antibodies and a second population of antibodies,
wherein said first population of antibodies is directed to a marker expressed by a population of cardiosphere-derived cells (CDCs),
wherein said second population of antibodies is directed to myosin light chain that is expressed by the damaged or diseased cardiac tissue of said subject; and
applying a magnetic field around or adjacent to the damaged or diseased cardiac tissue to enhance the targeting of said composition to the damaged or diseased cardiac tissue by counteracting the wash-out of said composition from the damaged or diseased cardiac tissue, thereby enhancing the interaction between said second population of antibodies and the myosin light chain expressed by said damaged or diseased cardiac tissue, thereby enhancing the delivery of said CDCs to said damaged or diseased cardiac tissue, and
wherein the enhanced delivery of said CDCs provides therapeutic improvements in said damaged or diseased cardiac tissue, thereby treating said damaged or diseased cardiac tissue.

3. The method of claim 2, wherein the first population of antibodies is directed to the CD34 marker on said CDCs.

4. The method of claim 2, wherein said cardiac tissue has been damaged by an acute adverse cardiac event.

5. The method of claim 4, wherein said acute adverse cardiac event comprises a myocardial infarction.

6. The method of claim 2, wherein the first population of antibodies is directed to the CD34 marker on said CDCs, wherein the cardiac tissue has been damaged by a myocardial infarction, wherein the magnetic particles comprise superparamagnetic iron oxide (SPIO) particles that are covalently linked to the two populations of antibodies, wherein the therapeutic composition is administered systemically, and wherein the applied magnetic field has a field strength of ranging from about 0.1 Tesla to about 100 Tesla.

7. The method of claim 2, wherein said damaged cardiac tissue results from chronic stress or disease of the heart comprising one or more of the following: chronic heart failure, systemic hypertension, pulmonary hypertension, valve dysfunction, congestive heart failure, and coronary artery disease.

8. The method of claim 2, wherein said therapeutic improvements comprise functional or anatomical repair of said damaged or diseased cardiac tissue.

9. The method of claim 8, wherein said therapeutic improvement comprises functional repair of said damaged or diseased tissue comprising an increase in cardiac output.

10. The method of claim 9, wherein said increase in cardiac output comprises an increase in left ventricular ejection fraction of at least 2%.

11. The method of claim 8, wherein said therapeutic improvement comprises anatomical repair of said damaged or diseased tissue comprising an increase in viable cardiac tissue.

12. The method of claim 11, wherein said therapeutic improvement comprises anatomical repair of said damaged or diseased tissue comprising an increase in cardiac wall thickness or a decrease in scar tissue formation.

13. The method of claim 2, wherein said magnetic particles are covalently coupled to said first and second populations of antibodies.

14. The method of claim 13, wherein said magnetic particles after coupling to said antibodies have a diameter of about 30 to 15000 nanometers.

15. A method for treating damaged cardiac tissue comprising:
administering to a first subject having damaged cardiac tissue, via a systemic delivery route, a therapeutic composition comprising:
magnetic particles covalently coupled to a first population of antibodies and a second population of antibodies,
wherein said first population of antibodies is directed to a marker expressed by a population of cardiosphere-derived cells (CDCs) isolated from a second subject,
wherein said second population of antibodies is directed to marker that is expressed by the damaged cardiac tissue of said subject; and
applying a magnetic field having a field strength of between about 0.1 to about 100 Tesla to the damaged cardiac tissue to counteract wash-out of said composition from the damaged cardiac tissue, thereby enhancing the delivery of said CDCs to said damaged cardiac tissue and treat said damaged cardiac tissue.

16. The method of claim 15, further comprising administering to the first subject an additional agent that reduces blood flow through the damaged cardiac tissue.

17. The method of claim 15, wherein the systemic delivery comprises intracoronary administration.

18. A method for treating damaged cardiac tissue comprising:
administering to a first subject, via a systemic delivery route, a magnetically responsive populations of therapeutic cells comprising cardiosphere-derived cells (CDCs) coupled to a magnetic particle comprising antibodies;
applying a magnetic field having a field strength of between about 0.1 to about 100 Tesla to the damaged cardiac tissue to enhance delivery of the magnetically responsive CDCs to said damaged cardiac tissue and treat said damaged cardiac tissue.

19. The method of claim 18, wherein the CDCs are obtained from a subject that is alloegeneic with respect to the first subject.

20. The method of claim 18, wherein the magnetic field is generated by an external magnet and wherein the enhanced delivery of said CDCs results in increased cardiac function or regeneration of cardiac tissue.
